(19) United States
(12) Patent Application Publication
(10) Pub. No.: US 2003/0215835 A1
(43) Pub. Date: Nov. 20, 2003

(54) Differentially-regulated prostate cancer genes

(76) Inventors: Zairen Sun, Rockville, MD (US); Gilbert Jay, North Bethesda, MD (US)

Correspondence Address: ORIGENE TECHNOLOGIES, INCORPORATED, 6 TAFT COURT, SUITE 100, ROCKVILLE, MD, 20850, US

(21) Appl. No.: 10/341,434

(22) Filed: Jan. 14, 2003

Related U.S. Application Data
(60) Provisional application No. 60/348,164, filed on Jan. 15, 2002.
(60) Provisional application No. 60/348,119, filed on Jan. 15, 2002.

(51) Int. Cl. C12Q 1/68; A61K 48/00; C12M 1/34

(52) U.S. Cl. 435/6; 514/44; 435/287.2

(57) ABSTRACT

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are differentially-regulated in prostate cancer and are therefore useful in variety of ways, including, but not limited to, as molecular markers, as drug targets, and for detecting, diagnosing, staging, monitoring, prognosticating, preventing or treating, determining predisposition to, etc., diseases and conditions, to prostate cancer.

This application claims the benefit of U.S. Provisional Application Serial Nos. 60/348,164 and 60/348,119, both filed Jan. 15, 2002, which are hereby incorporated by reference in their entirety.

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are differentially regulated in prostate cancer and are therefore useful in variety of ways, including, but not limited to, as molecular markers, as drug targets, and for detecting, diagnosing, staging, monitoring, prognosticating, preventing or treating, determining predisposition to, etc., diseases and conditions,, especially relating to prostate cancer. The identification of specific genes, and groups of genes, expressed in pathways physiologically relevant to prostate cancer permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

Prostate cancer is the most common form of cancer diagnosed in the American male, occurring predominantly in males over age 50. The number of men diagnosed with prostate cancer has steadily increased as a result of the increasing population of older men. The American Cancer Society estimates that in the year 2000, about 180,000 American men were diagnosed with prostate cancer and about 32,000 died from the disease. In comparison, 1998 estimates for lung cancer in men were 171,500 cases and 160,100 deaths, and for colorectal cancer, the estimates were 131,600 cases and 56,000 deaths. Despite these high numbers, 89 percent of men diagnosed with the disease will survive at least five years and 63 percent will survive at least 10 years.

Patients having prostate cancer display a wide range of phenotypes. In some men, following detection, the tumor remains a latent histological tumor and does not become clinically significant. However, in other men, the tumor progresses rapidly, metastasizing and killing the patient in a relatively short time. Prostate cancer can be cured if the tumor is confined to a small region of the gland and is discovered at early stage. In such cases, radiation or surgical removal often results in complete elimination of the disease. Frequently, however, the prostate cancer has already spread to surrounding tissue and metastasized to remote locations. In these cases, radiation and other therapies, are less likely to effect a complete cure.

Androgen deprivation is a conventional therapy to treat prostate cancer. Androgen blockade can be achieved through several different routes. Androgen suppressive drugs include, e.g., Lupron (leuprolide acetate), Casodex (bicalutamide), Eulexin (flutamide), Nilandron (nilutamide), Zoladex (goserelin acetate implant), and Viadur (leuprolide acetate), which act through several different mechanisms. While these drugs may offer remission and tumor regression in many cases, often the therapeutic effects are only temporary. Prostate tumors lose their sensitivity to such treatments, and become androgen-independent. Thus, new therapies are clearly needed.

The first clinical symptoms of prostate cancer are typically urinary disturbances, including painful and more frequent urination. Diagnosis for prostate cancer is usually accomplished using a combination of different procedures. Since the prostate is located next to the rectum, rectal digital examination allows the prostate to be examined manually for the presence of hyperplasia and abnormal tissue masses. Usually, this is the first line of detection. If a palpable mass is observed, a blood specimen can be assayed for prostate-specific antigen (PSA). Very little PSA is present in the blood of a healthy individual, but BPH and prostate cancer can cause large amounts of PSA to be released into the blood, indicating the presence of diseased tissue. Definitive diagnosis is generally accomplished by biopsy of the prostate tissue.

No single gene or protein has been identified which is responsible for the etiology of all prostate cancers. Although PSA is widely used as a diagnostic reagent, it has limitations in its sensitivity and its ability to detect early cancers. It is estimated that approximately 20% to 30% of tumors will be missed when PSA is used alone. It is likely that diagnostic and prognostic markers for prostate cancer disease will involve the identification and use of many different genes and gene products to reflect its multifactorial origin. With this in mind, combinations of the differentially-expressed genes of the present invention can be used as diagnostic and prognostic markers for prostate cancers.

A continuing goal is to characterize the gene expression patterns of the various prostate cancers to genetically differentiate them, providing important guidance in preventing, diagnosing, and treating cancers. Molecular pictures of cancer, such as the pattern of differentially-regulated genes identified herein, provide an important tool for molecularly dissecting and classifying cancer, identifying drug targets, providing prognosis and therapeutic information, etc. For instance, an array of polynucleotides corresponding to genes differentially regulated in prostate cancer can be used to screen tissue samples for the existence of cancer, to categorize the cancer (e.g., by the particular pattern observed), to grade the cancer (e.g., by the number of up- or down-regulated genes and their amounts of expression), to identify the source of a secondary tumor, to screen for metastatic cells, etc. These arrays can be used in combination with other markers, e.g., PSA, PMSA (prostate membrane specific antigen), or any of the grading systems used in clinical medicine.

As indicated by these studies, cancer is a highly diverse disease. Although all cancers share certain characteristics, the underlying cause and disease progression can differ significantly from patient to patient. So far, over a dozen distinct genes have been identified which, when mutant, result in a cancer. In breast cancer, alone, a handful of different genes have been isolated which either cause the cancer, or produce a predisposition to it. As a consequence, disease phenotypes for a particular cancer do not look all the same. In addition to the differences in the gene(s) responsible for the cancer, heterogeneity among individuals, e.g., in age, health, sex, and genetic background, can also influence the disease and its progression. Gene penetrance, in particular, can vary widely among population members. Recent studies have shown tremendous diversity in gene expression patterns among cancer patients. For these and other reasons, one gene/polypeptide target alone can be insufficient to diagnose or treat a cancer. Even a gene which is highly differentially-expressed and penetrant in cancer patients may not be so highly expressed in all patients and at all stages of the cancer. By selecting a set of genes and/or the polypeptides they encode, cancer diagnostics and therapeutics can be designed which effectively diagnose and treat a population of diseased individuals, rather than only a small handful when single genes are targeted.

Table 1 is a list of genes up-regulated in prostate cancer; Table 2 lists genes down-regulated. The column at the far left is the gene's alphanumeric designation. “GI#” indicates the accession number of the gene. “Classification” is the cellular localization of the gene product: membrane (e.g., a cell-surface molecule), secreted, intracellular, or nuclear. The characterization of the gene under the “description” heading is based on listing in GenBank. The nucleotide and amino acid sequences of the gene can be retrieved routinely from Genbank, e.g., by searching the accession number. These sequences, and all information referenced to the accession number, are incorporated by reference in their entirety. The numbers in the first column refer to nucleotide and amino acid sequence, respectively.

The polypeptide sequences was analyzed for the presence of functional domains using the publicly available Pfam program. Domains present in each polypeptide are listed under “domain.” Any abbreviations are those used in Pfam. The start of the domain is indicated by “seq-f” and the end of the domain by “seq-t.” The “score” is the statistical score of this match to the domain in bits. In general, a higher score indicates a better match. “E” is the statistical score of this match in Evalue (frequentist) approach. The smaller score in this case shows a better match between the domain and the query sequence. For more information on the program and scoring, see, e.g., Sonnhammer et al., Proteins: Structure, Function and Genetics 28:405-420 (1997); Sonnhammer et al., Nucleic Acids Research 26:320-322 (1998); Bateman et al., Nucleic Acids Research 27:260-262 (1999); Bateman et al., Nucleic Acids Research 28:263-266 (2000).

Nucleic Acids

In accordance with the present invention, genes have been identified which are differentially expressed in prostate cancer. Tables 1 and list genes which are differentially-regulated in the cancer. These genes can be further divided into groups based on additional characteristics of their expression and the tissues in which they are expressed. For instance, genes can be further subdivided based on the stage and/or grade of the cancer in which they are expressed. Genes can also be grouped based on their penetrance in a prostate cancer, e.g., expressed in all prostate cancer examined, expressed in a certain percentage of prostate cancer examined, etc. Additionally, genes can be categorized by their function and/or the polypeptides they encode. This includes, but is not limited to, cellular localization, functional activity (e.g., kinase, cytoskeletal element, or transcriptional factor), functional pathway (e.g., protein manufacture, cell signaling, cell movement, cell adhesion, responsivity to cAMP, energy production, etc.), etc. These groupings do not restrict or limit the use such genes in therapeutic, diagnostic, prognostic, etc., applications. For instance, a gene which is expressed in only some cancers (e.g., incompletely penetrant) may be useful in therapeutic applications to treat a subset of cancers. Similarly, a co-penetrant gene, or a gene which is expressed in prostate cancer and other normal tissues, may be useful as a therapeutic or diagnostic, even if its expression pattern is not highly prostate specific. Thus, the uses of the genes or their products are not limited by their patterns of expression.

In developing reagents for the diagnosis and treatment of a disease, it may be useful to know the cellular localization of a differentially expressed polypeptide to determine how to use it as a target. Proteins which are secreted or on the cell-surface are more readily accessible than intracellular proteins, and can be, e.g., blocked or inhibited to restore levels to normal.

In recent years, there have been numerous reports on specific targeting of tumor cells with monoclonal antibody-drug conjugates using cell-surface proteins. See, e.g., Chari., Adv. Drug Deliv. Res., 31: 89-104 (1998); Pietersz and Krauer, J. Drug Targeting, 2: 183-215 (1994); Sela et al., in Immunoconjugates, 189-216 (C. Vogel, ed. 1987); Ghose et al., in Targeted Drugs, 1-22 (E. Goldberg, ed. 1983); Diener et al., in Antibody mediated delivery systems, 1-23 (J. Rodwell, ed. 1988); Pietersz et al., in Antibody mediated delivery systems, 25-53 (J. Rodwell, ed. 1988); Bumol et al., in Antibody mediated delivery system, 55-79 (J. Rodwell, ed. 1988). Cytotoxic drugs such as methotrexate, daunorubicin, doxorubicin, vincristine, vinblastine, melphalan, mitomycin C, and chlorambucil have been conjugated to a variety of monoclonal antibodies. Therapeutic agents can be directly conjugated to the antibody, or through cleavable linkers which facilitate the release of the agent in active form only when it is inside the cell. See, e.g., U.S. Pat. No. 6,333,410.

By the phrase “differential expression,” it is meant that the levels of expression of a gene, as measured by its transcription or translation product, are different depending upon the specific cell-type or tissue (e.g., in an averaging assay that looks at a population of cells). There are no absolute amounts by which the gene expression levels must vary, as long as the differences are measurable.

The phrase “up-regulated” indicates that an mRNA transcript or other nucleic acid corresponding to a polynucleotide of the present invention is expressed in larger amounts in a cancer as compared to the same transcript expressed in normal cells from which the cancer was derived. The phrase “down-regulated” indicates that an mRNA transcript or other nucleic acid corresponding to a polynucleotide of the present invention is expressed in lower amounts in a cancer as compared to the same transcript expressed in normal cells from which the cancer was derived. In general, differential-regulation can be assessed by any suitable method, including any of the nucleic acid detection and hybridization methods mentioned below, as well as polypeptide-based methods. Up-regulation also includes going from substantially no expression in a normal tissue, from detectable expression in a normal tissue, from significant expression in a normal tissue, to higher levels in the cancer. Down-regulation also includes going from substantially no expression in a normal tissue, from detectable expression in a normal tissue, from significant expression in a normal tissue, to higher levels in the cancer.

Differential regulation can be determined by any suitable method, e.g., by comparing its abundance per gram of RNA (e.g., total RNA, polyadenylated mRNA, etc.) extracted from a prostate tissue in comparison to the corresponding normal tissue. The normal tissue can be from the same or different individual or source. For convenience, it can be supplied as a separate component or in a kit in combination with probes and other reagents for detecting genes. The quantity by which a nucleic acid is differentially-regulated can be any value, e.g., about 10% more or less of normal expression, about 50% more or less of normal expression, 2-fold more or less, 5-fold more or less, 10-fold more or less, etc.

The amount of transcript can also be compared to a different gene in the same sample, especially a gene whose abundance is known and substantially no different in its expression between normal and cancer cells (e.g., a “control” gene). If represented as a ratio, with the quantity of differentially-regulated gene transcript in the numerator and the control gene transcript in the denominator, the ratio would be larger, e.g., in prostate cancer than in a sample from normal prostate tissue.

Differential-regulation can arise through a number of different mechanisms. The present invention is not bound by any specific way through which it occurs. Differential-regulation of a polynucleotide can occur, e.g., by modulating (1) transcriptional rate of the gene (e.g., increasing its rate, inducing or stimulating its transcription from a basal, low-level rate, etc.), (2) the post-transcriptional processing of RNA transcripts, (3) the transport of RNA from the nucleus into the cytoplasm, (4) RNA nuclear and cytoplasmic turnover, and polypeptide turnover (e.g., by virtue of having higher stability or resistance to degradation), and combinations thereof. See, e.g., Tollervey and Caceras, Cell, 103:703-709, 2000.

A differentially-regulated polynucleotide is useful in a variety of different applications as described in greater details below. Because it is more abundant in cancer, it and its expression products can be used in a diagnostic test to assay for the presence of cancer, e.g., in tissue sections, in a biopsy sample, in total RNA, in lymph, in blood, etc. Differentially-regulated polynucleotides and polypeptides can be used individually, or in groups, to assess the cancer, e.g., to determine the specific type of cancer, its stage of development, the nature of the genetic defect, etc., or to assess the efficacy of a treatment modality. How to use polynucleotides in diagnostic and prognostic assays is discussed below. In addition, the polynucleotides and the polypeptides they encode, can serve as a target for therapy or drug discovery. A polypeptide, coded for by a differentially-regulated polynucleotide, which is displayed on the cell-surface, can be a target for immunotherapy to destroy, inhibit, etc., the diseased tissue. Differentially-regulated transcripts can also be used in drug discovery schemes to identify pharmacological agents which modulate, suppress, inhibit, activate, increase, etc., their differential-regulation, thereby preventing the phenotype associated with their expression. Thus, a differentially-regulated polynucleotide and its expression products of the present invention have significant applications in diagnostic, therapeutic, prognostic, drug development, and related areas.

The expression patterns of the selectively expressed genes disclosed herein can be described as a “fingerprint” in that they are a distinctive pattern displayed by a tissue. Just as with a fingerprint, an expression pattern can be used as a unique identifier to characterize the status of a tissue sample. The list of expressed sequences disclosed herein provides an example of such a tissue expression profile. It can be used as a point of reference to compare and characterize samples. Tissue fingerprints can be used in many ways, e.g., to classify a tissue as prostate cancer, to determine the origin of a metastatic cells, to assess the physiological status of a tissue, to determine the effect of a particular treatment regime on a tissue, and to evaluate the toxicity of a compound on a tissue of interest, to determine the presence of a cancer in a biopsy sample, to assess the efficacy of a cancer therapy in a human patient or a non-human animal model, to detect circulating cancer cells in blood or a lymph node biopsy, etc. While the expression profile of the complete gene set represented in Tables 1 and 2 may be most informative, a fingerprint containing expression information from less than the full collection can be useful, as well. In the same way that an incomplete fingerprint may contain enough of the pattern of whorls, arches, loops, and ridges, to identify the individual, a cell expression fingerprint containing less than the full complement may be adequate to provide useful and unique identifying and other information about the sample. Moreover, cancer is a multifactorial disease, involving genetic aberrations in more than gene locus. This multifaceted nature may be reflected in different cell expression profiles associated with prostate cancers arising in different individuals, in different locations in the same individual, or even within the same cancer locus. As a result, a complete match with a particular cell expression profile, as shown herein, is not necessary to classify a cancer as being of the same type or stage. Similarity to one cell expression profile, e.g., as compared to another, can be adequate to classify cancer types, grades, and stages.

For example, the tissue-selective genes disclosed herein represent the configuration of genes expressed by a normal tissue. To determine the effect of a toxin on a tissue, a sample of tissue is obtained prior to toxin exposure (“control”) and then at one or more time points after toxin exposure (“experimental”). An array of tissue-selective probes can be used to assess the expression patterns for both the control and experimental samples. Methods of making and using arrays are described below.

A mammalian polynucleotide, or fragment thereof, of the present invention is a polynucleotide having a nucleotide sequence obtainable from a natural source. When the species name is used, e.g., human, it indicates that the polynucleotide or polypeptide is obtainable from a natural source. It therefore includes naturally-occurring normal, naturally-occurring mutant, and naturally-occurring polymorphic alleles (e.g., SNPs), differentially-spliced transcripts, splice-variants, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc. The polynucleotides described in Tables 1 and 2 are reference to specific nucleotide sequences as listed in GenBank. The present invention relates to these sequences, as well as any naturally-occurring variants and polymorphisms. A polynucleotide which “codes without interruption” refers to a polynucleotide having a continuous open reading frame (“ORF”) as compared to an ORF which is interrupted by introns or other noncoding sequences.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions can be identified upstream of coding and expressed RNAs, and assayed routinely for activity, e.g., by joining to a reporter gene (e.g., CAT, GFP, alkaline phosphatase, luciferase, galatosidase). A promoter obtained from a prostate cancer can be used, e.g., in gene therapy to obtain tissue-specific expression of a heterologous gene (e.g., coding for a therapeutic product or cytotoxin).

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR54 0, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc.

The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in Tables 1 and 2, and genomic sequences thereof. A nucleotide sequence hybridizing to the latter sequence will have a complementary polynucleotide strand, or act as a template for one in the presence of a polymnerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in Tables 1 and 2 and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in Tables 1 and 2 or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6× SSC, 0.5% SDS, 100 μg/ml denatured salmon sperm DNA, 5× Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a polynucleotide probe in a hybridization solution containing, e.g., about 5× SSC, 0.5% SDS, 100 μg/ml denatured salmon sperm DNA and 50% formamide, at 42° C.; or, at 42° C., or hybridizing at 42° C. in 5× 42° C. SSPE, 0.5% SDS, and 50% formamide, 100 mg/ml denatured salmon sperm DNA, and washing at 65° C. in 0.1% SSC and 0.1% SDS. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than, e.g., less than 10%, 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al.. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log₁₀[Na⁺]+0.41(% GC)−600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, or 97%, nucleotide complementarity between the probe (e.g., a short polynucleotide of Table 1 or 2) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. etc. BLAST can be used to calculate amino acid sequence identity, amino acid sequence homology, and nucleotide sequence identity. These calculations can be made along the entire length of each of the target sequences which are to be compared.

After two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity=100[1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull. Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence of Tables 1 and 2, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about seven nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8-400, 8-300, 8-150, 8-100, 8-75, 7-50, 10-25, 14-16, at least about 8, at least about 10, 11, 12, 13, 14, 15, 16, 17, 18, 19, 20, 21, 22, 23, 24, at least about 15, at least about 25, 26, or more.

The polynucleotides can have non-naturally-occurring nucleotides, e.g., inosine, AZT, 3TC, etc. The polynucleotides can have 100% sequence identity or complementarity to a sequence of Table 1, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for differentially-regulated genes of the present invention, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample and distinguish them from non-target genes. It is specific in the sense that it can be used to detect polynucleotides above background noise (“non-specific binding”). A specific sequence is a defined order of nucleotides (or amino acid sequences, if it is a polypeptide sequence) which occurs in the polynucleotide, e.g., in the nucleotide sequences selected from Tables 1 and 2 , and which is characteristic of that target sequence, and substantially no non-target sequences.

A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complementarity) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of prostate is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, ssRNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting differentially-regulated genes of the present invention. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Detection can be accomplished in combination with polynucleotide probes for other genes, e.g., genes which are expressed in other disease states, tissues, cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, peripheral blood cells (T-cells, lymphocytes, etc.), embryo, normal, fat, adult and embryonic stem cells, specific cell-types, such as endothelial, epithelial, etc.

Polynucleotides can be used in wide range of methods and compositions, including for detecting, diagnosing, staging, grading, assessing, prognosticating, etc. diseases and disorders associated with differentially-regulated genes of the present invention, for monitoring or assessing therapeutic and/or preventative measures, in ordered arrays, etc. Any method of detecting genes and polynucleotides of Tables 1 and 2 can be used; certainly, the present invention is not to be limited how such methods are implemented.

Along these lines, the present invention relates to methods of detecting differentially-regulated genes described herein in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is selected from Tables 1 and 2, a polynucleotide having, e.g., about 70%, 80%, 85%, 90%, 95%, 99%, or more sequence identity thereto, effective or specific fragments thereof, or complements thereto. The detection method can be applied to any sample, e.g., cultured primary, secondary, or established cell lines, tissue biopsy, blood, urine, stool, and other bodily fluids, for any purpose.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a polynucleotide set forth in Table 1 is desired, a probe can be selected which can hybridize to such target gene under high stringent conditions, without significant hybridization to other genes in the sample. To detect homologs of a polynucleotide set forth in Tables 1 and 2, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples.. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

Methods of Identifying Polymorphisms, Mutations, etc., of a Differentially-Regulated Gene

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from cancers that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from Table 1 or 2 is used as a probe. The selected mutant alleles, SNPs, polymorphisms, etc., can be used diagnostically to determine whether a subject has, or is susceptible to a disorder associated with a differentially-regulated gene, as well as to design therapies and predict the outcome of the disorder. Methods involve, e.g., diagnosing a disorder associated with a differentially-regulated gene or determining susceptibility to a disorder, comprising, detecting the presence of a mutation in a gene represented by a polynucleotide selected from Table 1 or 2. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

The present invention also relates to methods of detecting polymorphisms in a differentially-regulated gene, comprising, e.g., comparing the structure of: genomic DNA comprising all or part of said gene, mRNA comprising all or part of said gene, cDNA comprising all or part of said gene, or a polypeptide comprising all or part of said gene, with the structure of said gene as set forth herein. The methods can be carried out on a sample from any source, e.g., cells, tissues, body fluids, blood, urine, stool, hair, egg, sperm, etc.

These methods can be implemented in many different ways. For example, “comparing the structure” steps include, but are not limited to, comparing restriction maps, nucleotide sequences, amino acid sequences, RFLPs, DNAse sites, DNA methylation fingerprints (e.g., U.S. Pat. No. 6,214,556), protein cleavage sites, molecular weights, electrophoretic mobilities, charges, ion mobility, etc., between a standard gene and a test gene. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Polynucleotide Expression, Polypeptides Produced Thereby, and Specific-Binding Partners Thereto

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, PC-3 (CRL-1435), LNCaP (CRL-1740), CA-HPV-10 (CRL-2220), PZ-HPV-7 (CRL-2221), MDA-PCa 2b (CRL-2422), 22Rv1 (CRL2505), NCI-H660 (CRL-5813), HS 804.Sk (CRL-7535), LNCaP-FGF (CRL-10995), RWPE-1 (CRL-11609), RWPE-2 (CRL-11610), PWR-1E (CRL 11611), rat MAT-Ly-LuB-2 (CRL-2376), and other prostate cells, insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in Table 1 or 2, or it can contain degenerate codons kcoding for the same amino acid sequences. For instance, it may be desirable to change the codons in the sequence to optimize the sequence for expression in a desired host. See, e.g., U.S. Pat. Nos. 5,567,600 and 5,567,862. A polypeptide according to the present invention can be recovered from natural sources, transformed host cells (culture medium or cells) according to the usual methods.

The present invention also relates to specific-binding partners. These include antibodies which are specific for polypeptides encoded by polynucleotides of the present invention, as well as other binding-partners which interact with polynucleotides and polypeptides of the present invention. Protein-protein interactions between polypeptides of Tables 1 or 2, and other polypeptides and binding partners can be identified using any suitable methods, e.g., protein binding assays (e.g., filtration assays, chromatography, etc.), yeast two-hybrid system (Fields and Song, Nature, 340: 245-247, 1989), protein arrays, gel-shift assays, FRET (fluorescence resonance energy transfer) assays, etc. Nucleic acid interactions (e.g., protein-DNA or protein-RNA) can be assessed using gel-shift assays, e.g., as carried out in U.S. Pat. Nos. 6,333,407 and 5,789,538.

Antibodies, e.g., polyclonal, monoclonal, recombinant, chimeric, humanized, single-chain, Fab, and fragments thereof, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can be IgM, IgG, subtypes, IgG2a, IgG1, etc. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, chicken (e.g., IgY; see, Duan, WO/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA, can be prepared against specific epitopes or domains of differentially regulated genes. The preparation of polyclonal, monoclonal, single-chain, antibody fragments, and other antibody types and forms are well-known to those skilled in the art.

Methods of Detecting Polypeptides

Polypeptides coded for by a differentially-regulated gene of the present invention can be detected, visualized, determined, quantitated, etc. according to any effective method. Useful methods include, e.g., but are not limited to, immunoassays, RIA (radioimimunoassay), ELISA, (enzyme-linked-immunosorbent assay), immunofluorescence, flow cytometry, histology, electron microscopy, light microscopy, in situ assays, immunoprecipitation, Western blot, etc.

Immunoassays may be carried in liquid or on biological support. For instance, a sample (e.g., blood, stool, urine, cells, tissue, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled differentially-regulated gene specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, t in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, .beta.-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect differentially-regulated peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Tissue and Disease

The prostate is a secretory organ surrounding the neck of the bladder and urethra. Its primary function is to produce fluids and other materials necessary for sperm transport and maintenance. Structurally, it has both glandular and nonglandular components. The glandular component is predominantly comprised of ducts and acini responsible for the production and transport prostatic fluids. Epithelial cells are the main identifiable cell found in these regions, primarily of the basal and secretory types, but also endocrine-paracrine and transitional epithelial. The non-glandular component contains the capsular and muscle tissues, which, respectively, hold the organ together and function in fluid discharge. See, e.g., Histology for Pathologists, Sternberg, S. S., editor, Raven Press, NY, 1992, Chapter 40.

The major diseases of the prostate include, e.g., prostatic hyperplasia (BPH), prostatitis, and prostate cancer (e.g., prostatic adenocarcinoma). BPH is a benign, proliferative disease of the prostatic epithelial cells. While it may cause urinary tract obstruction in some patients, for the most part, it is generally asymptomatic. Prostate cancer, on the other hand, is the most common form of cancer in white males in the United States, occurring predominantly in males over age 50. The prevalence of prostate diseases, such as prostate cancer, has made the discovery of prostate selective markers and gene expression patterns of great importance.

The most common scale of assessing prostate pathology is the Gleason grading system. See, e.g., Bostwick, Am. J Clin. Path., 102: s38-s56, 1994. Once the cancer is identified, staging can assess the size, location, and extent of the cancer. Several different staging scales are commonly used, including stages A-D, and Tumor-Nodes-Metastases (TNM). For treatment, diagnosis, staging, etc., of prostate conditions, methods can be carried out analogously to, and in combination with, U.S. Pat. Nos. 6,107,090; 6,057,116; 6,034,218; 6,004,267; 5,919,638; 5,882,864; 5,763,202; 5,747,264; 5,688,649; 5,552,277.

In addition, the present invention relates to methods of assessing a therapeutic or preventative intervention in a subject having a prostate cancer, comprising, e.g., detecting the expression levels of up-regulated target genes, wherein the target genes comprise a gene which is represented by a sequence selected from Table 1 or 2, or, a gene represented by a sequence having 95% sequence identity or more to a sequence selected from Table 1 or 2. By “therapeutic or preventative intervention,” it is meant, e.g., a drug administered a patient, surgery, radiation, chemotherapy, and other measures taken to prevent a cancer or treat a cancer.

Grading, Staging, Comparing, Assessing, Methods and Compositions

The present invention also relates to methods and compositions for staging and grading cancers. As already defined, staging relates to determining the extent of a cancer's spread, including its size and the degree to which other tissues, such as lymph nodes are involved in the cancer. Grading refers to the degree of a cell's retention of the characteristics of the tissue of its origin. A lower grade cancer comprises tumor cells that more closely resemble normal cells than a medium or higher grade cancer. Grading can be a useful diagnostic and prognostic tool. Higher grade cancers usually behave more aggressively than lower grade cancers. Thus, knowledge of the cancer grade, as well as its stage, can be a significant factor in the choice of the appropriate therapeutic intervention for the particular patient, e.g., surgery, radiation, chemotherapy, etc. Staging and grading can also be used in conjunction with a therapy to assess its efficacy, to determine prognosis, to determine effective dosages, etc.

Various methods of staging and grading cancers can be employed in accordance with the present invention. A “cell expression profile” or “cell expression fingerprint” is a representation of the expression of various different genes in a given cell or sample comprising cells. These cell expression profiles can be useful as reference standards. The cell expression fingerprints can be used alone for grading, or in combination with other grading methods.

The present invention also relates to methods and compositions for diagnosing a prostate cancer, or determining susceptibility to a prostate cancer, using polynucleotides, polypeptides, and specific-binding partners of the present invention to detect, assess, determine, etc., differentially-regulated genes of the present invention. In such methods, the gene can serve as a marker for prostate cancer, e.g., where the gene, when mutant, is a direct cause of the prostate cancer; where the gene is affected by another gene(s) which is directly responsible for the prostate cancer, e.g., when the gene is part of the same signaling pathway as the directly responsible gene; and, where the gene is chromosomally linked to the gene(s) directly responsible for the prostate cancer, and segregates with it. Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The present invention relates to methods of diagnosing a disorder associated with prostate cancer, or determining a subject's susceptibility to such prostate cancer, comprising, e.g., assessing the expression of a differentially-regulated gene in a tissue sample comprising tissue or cells suspected of having the [(e.g., where the sample comprises prostate). The phrase “diagnosing” indicates that it is determined whether the sample has a prostate cancer cells. “Determining a subject's susceptibility to a prostate cancer” indicates that the subject is assessed for whether s/he is predisposed to get-such a disease or disorder, where the predisposition is indicated by abnormal expression of the gene (e.g., gene mutation, gene expression pattern is not normal, etc.). Predisposition or susceptibility to a disease may result when a such disease is influenced by epigenetic, environmental, etc., factors.

By the phrase “assessing expression of a differentially-regulated gene,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a normal gene, e.g., a gene which is not associated with the disorder. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc. Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

The genes and polypeptides of the present invention can be used to identify, detect, stage, determine the presence of, prognosticate, treat, study, etc., diseases and conditions of the prostate mentioned above. The present invention relates to methods of identifying a genetic basis for a disease or disease-susceptibility, comprising, e.g., determining the association of a prostate cancer or cancer-susceptibility with a gene of the present invention. An association between a disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target. Any region of the gene can be used as a source of the DNA marker, exons, introns, intergenic regions, etc.

Human linkage maps can be constructed to establish a relationship between a gene and prostate cancer. Typically, polymorphic molecular markers (e.g., STRP's, SNP's, RFLP's, VNTR's) are identified within the region, linkage and map distance between the markers is then established, and then linkage is established between phenotype and the various individual molecular markers. Maps can be produced for an individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype. See, e.g., Kruglyak et al., Am. J. Hum. Genet., 58, 1347-1363, 1996; Matise et al., Nat. Genet., 6(4):384-90, 1994.

Assessing the effects of therapeutic and preventative interventions (e.g., administration of a drug, chemotherapy, radiation, etc.) on prostate cancer is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving cancer treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having prostate cancer, or, susceptible to such a disorder, comprising, e.g., detecting the expression levels of one or more differentially-regulated genes of the present invention. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose prostate cancer.

Expression can be assessed in any sample comprising any tissue or cell type, body fluid, etc., as discussed for other methods of the present invention, including cells from prostate can be used, or cells derived from prostate. By the phrase “cells derived from prostate,” it is meant that the derived cells originate from prostate, e.g., when metastasis from a primary tumor site has occurred, when a progenitor-type or pluripotent cell gives rise to other cells, etc.

The present invention also relates to methods of using polypeptide binding partners, such as antibodies, to deliver active agents to the prostate for different purposes, including, e.g., for diagnostic, therapeutic (e.g., to treat prostate), and research purposes. Methods can involve delivering or administering an active agent to the prostate, comprising, e.g., administering to a subject in need thereof, an effective amount of an active agent coupled to a binding partner specific for human polypeptide selected from Tables 1 and 2, wherein said binding partner is effective to deliver said active agent specifically to prostate.

Any type of active agent can be used in combination, including, therapeutic, cytotoxic, cytostatic, chemotherapeutic, anti-neoplastic, anti-proliferative, anti-biotic, etc., agents. A chemotherapeutic agent can be, e.g., DNA-interactive agent, alkylating agent, antimetabolite, tubulin-interactive agent, hormonal agent, hydroxyurea, Cisplatin, Cyclophosphamide, Altretamine, Bleomycin, Dactinomycin, Doxorubicin, Etoposide, Teniposide, paclitaxel, cytoxan, 2-methoxycarbonylaminobenzimidazole, Plicamycin, Methotrexate, Fluorouracil, Fluorodeoxyuridin, CB3717, Azacitidine, Floxuridine, Mercapyopurine, 6-Thioguanine, Pentostatin, Cytarabine, Fludarabine, etc. Agents can also be contrast agents useful in imaging technology, e.g., X-ray, CT, CAT, MRI, ultrasound, PET, SPECT, and scintographic.

An active agent can be associated in any manner with a binding partner which is effective to achieve its delivery specifically to the target. Specific delivery or targeting indicates that th the active agent is in a liposome, or other carrier, and the binding partner is associated with the liposome surface. The binding partner can be oriented in such a way that it is able to bind to its target, e.g., on the cell surface. Methods for delivery of DNA via a cell-surface receptor is described, e.g., in U.S. Pat. No. 6,339,139.

Identifying Agent Methods

The present invention also relates to methods of identifying agents, and the agents themselves, which modulate prostate cancer genes. These agents can be used to modulate the biological activity of the polypeptide encoded for the gene, or the gene, itself. Agents which regulate the gene or its product are useful in variety of different environments, including as medicinal agents to treat or prevent disorders associated with prostate cancer genes and as research reagents to modify the function of tissues and cell.

Methods of identifying agents generally comprise steps in which an agent is placed in contact with the gene, transcription product, translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of a prostate cancer gene, a method can comprise, in any effective order, one or more of the following steps, e.g., contacting a prostate cancer gene (e.g., in a cell population) with a test agent under conditions effective for said test agent to modulate the expression of the prostate cancer, and determining whether said test agent modulates said gene. An agent can modulate expression of a gene at any level, including transcription, translation, and/or perdurance of the nucleic acid (e.g., degradation, stability, etc.) in the cell.

For modulating the biological activity of prostate cancer polypeptides, a method can comprise, in any effective order, one or more of the following steps, e.g., contacting a polypeptide (e.g., in a cell, lysate, or isolated) with a test agent under conditions effective for said test agent to modulate the biological activity of said polypeptide, and determining whether said test agent modulates said biological activity.

Contacting a gene or polypeptide with the test agent can be accomplished by any suitable method and/or means that places the agent in a position to functionally control its expression or biological activity. Functional control indicates that the agent can exert its physiological effect on the gene or polypeptide through whatever mechanism it works. The choice of the method and/or means can depend upon the nature of the agent and the condition and type of environment in which the gene or polypeptide is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

Agents can be directed to, or targeted to, any part of the polypeptide which is effective for modulating it. For example, agents, such as antibodies and small molecules, can be targeted to cell-surface, exposed, extracellular, ligand binding, functional, etc., domains of the polypeptide. Agents can also be directed to intracellular regions and domains, e.g., regions where the polypeptide couples or interacts with intracellular or intramembrane binding partners.

After the agent has been administered in such a way that it can gain access to the gene or polypeptide, it can be determined whether the test agent modulates the gene or polypeptide expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc.

A test agent can be of any molecular composition, e.g., chemical compounds, biomolecules, such as polypeptides, lipids, nucleic acids (e.g., antisense to a polynucleotide sequence selected from Table 1 or 2), carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity a polypeptide in a lysate or other cell-free form. Antisense can also be used as test agents to modulate gene expression.

Markers The polynucleotides of the present invention can be used with other markers, especially prostate and prostate cancer markers to identity, detect, stage, diagnosis, determine, prognosticate, treat, etc., tissue, diseases and conditions, etc, of the prostate. Markers can be polynucleotides, polypeptides, antibodies, ligands, specific binding partners, etc.

A number of genes and gene products have been identified which are associated with prostate cancer metastasis and/or progression, e.g., PSA, KAII (shows decreased expression in metastatic cells; Dong et al., Science, 268:884-6, 1995), D44 isoforms (differentially-regulated during carcinoma progression; Noordzij et al., Clin. Cancer Res., 3:805-15, 1997), p53 (Effert et al., J. Urol., 150:257-61, 1993), Rb, CDKN2, E-cadherin, PTEN (Hamilton et al., Br. J Cancer, 82:1671-6, 2000; Dong et al., Clin. Cancer Res., 7:304-308, 2001), bcl-2, prostatic acid phosphatase (PAP), prostate specific membrane antigen (e.g., U.S. Pat. Nos. 5,538,866 and 6,107,090), Smad3 (e.g., Kang et al., Proc. Natl. Acad. Sci., 98:3018-3023, 2001), TGF-beta, and other oncogenes and tumor suppressor genes. See, also, Myers and Grizzle, Eur. Urol., 30:153-166, 1996, for other biomarkers associated with prostatic carcinoma, such as PCNA, p185-erbB-2, p180erbB-3, TAG-72, nm23-H1 and FASE. Such markers can be used in combination with the methods of the present invention to facilitate identifying, grading, staging, prognostication, etc, of conditions and diseases of the prostate.

Therapeutics

Selective polynucleotides, polypeptides, and specific-binding partners thereto, can be utilized in therapeutic applications, especially to treat prostate cancer. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a tissue-specific antigen can be used to stimulate the body to destroy or attack the cancer, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well.

In addition to immunotherapy, polynucleotides and polypeptides can be used as targets for non-immunotherapeutic applications, e.g., using compounds which interfere with function, expression (e.g., antisense as a therapeutic agent), assembly, etc. RNA interference can be used in vivtro and in vivo to silence differentially-expressed genes when its expression contributes to a disease (but also for other purposes, e.g., to identify the gene's function to change a developmental pathway of a cell, etc.). See, e.g., Sharp and Zamore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc. Therapeutic agents of the present invention can be administered in any form by any effective route, including, e.g., oral, parenteral, enteral, intraperitoneal, topical, transdermal (e.g., using any standard patch), ophthalmic, intravenously, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive.

In addition to therapeutics, per se, the present invention also relates to methods of treating prostate cancer showing altered expression of differentially-regulated genes of Tables 1 and 2, comprising, e.g., administering to a subject in need thereof a therapeutic agent which is effective for regulating expression of said genes and/or which is effective in treating said disease. The term “treating” is used conventionally, e.g., the management or care of a subject for the purpose of combating, alleviating, reducing, relieving, improving the condition of, etc., of a disease or disorder. By the phrase “altered expression,” it is meant that the disease is associated with a mutation in the gene, or any modification to the gene (or corresponding product) which affects its normal function. Thus, expression of a differentially-regulated gene refers to, e.g., transcription, translation, splicing, stability of the mRNA or protein product, activity of the gene product, differential expression, etc.

Any agent which “treats” the disease can be used. Such an agent can be one which regulates the expression of the gene. Expression refers to the same acts already mentioned, e.g. transcription, translation, splicing, stability of the mRNA or protein product, activity of the gene product, differential expression, etc. For instance, if the condition was a result of a complete deficiency of the gene product, administration of gene product to a patient would be said to treat the disease and regulate the gene's expression. Many other possible situations are possible, e.g., where the gene is aberrantly expressed, and the therapeutic agent regulates the aberrant expression by restoring its normal expression pattern.

Antisense

Antisense polynucleotide (e.g., RNA) can also be prepared from a polynucleotide according to the present invention, preferably an anti-sense to a sequence of Table 1 or 2. Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708. An antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635.

Arrays

The present invention also relates to an ordered array of polynucleotide probes and specific-binding partners (e.g., antibodies) for detecting the expression of differentially-regulated genes in a sample, comprising, one or more polynucleotide probes or specific binding partners associated with a solid support, wherein each probe is specific for said genes, and the probes comprise nucleotide sequences selected from Table 1 and 2 which is specific for said gene, a nucleotide sequence having sequence identity to polynucleotide of Table 1 or 2 which is specific for said gene or polynucleotide, or complements thereto, or a specific-binding partner which is specific for said genes.

The phrase “ordered array” indicates that the probes are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Ordered arrays can further comprise polynucleotide probes or specific-binding partners which are specific for other genes, including genes specific for prostate or disorders associated with prostate, such as prostate cancer.

Transgenic Animals

The present invention also relates to transgenic animals comprising differentially-regulated No. 6,242,667). The term “gene” as used herein includes any part of a gene, i.e., regulatory sequences, promoters, enhancers, exons, introns, coding sequences, etc. The nucleic acid present in the construct or transgene can be naturally-occurring wild-type, polymorphic, or mutated. Where the animal is a non-human animal, its homolog can be used instead. Transgenic animals can be susceptible to prostate cancer.

Along these lines, polynucleotides of the present invention can be used to create transgenic animals, e.g. a non-human animal, comprising at least one cell whose genome comprises a functional disruption of a differentially-regulated gene (e.g., a mouse homolog when a mouse is used). By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced. The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the a differentially-regulated gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of its a differentially-regulated gene has been “knocked-out.” Knock-outs can be homozygous or heterozygous.

For creating functional disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, a differentially-regulated gene locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into a a differentially-regulated gene exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

A transgenic animal, or animal cell, lacking one or more functional differentially-regulated genes can be useful in a variety of applications, including, as an animal model for cancer, for drug screening assays, as a source of tissues deficient in said gene activity, and any of the utilities mentioned in any issued U.S. patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824.

The present invention also relates to non-human, transgenic animal whose genome comprises recombinant a differentially-regulated gene nucleic acid operatively linked to an expression control sequence effective to express said coding sequence, e.g., in prostate. such a transgenic animal can also be referred to as a “knock-in” animal since an exogenous gene has been introduced, stably, into its genome.

A recombinant a differentially-regulated gene nucleic acid refers to a gene which has been introduced into a target host cell and optionally modified, such as cells derived from animals, plants, bacteria, yeast, etc. A recombinant a differentially-regulated gene includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, the recombinant a differentially-regulated gene has been stably integrated into the genome of the animal. The a differentially-regulated gene nucleic acid in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11:1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P.C., et al., “Tissue- and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at 1oxP-Containing DNA sequences placed into the mammalian genome,” Polynucleotides Research, 17(l):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, New York, 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving gene sequences from a computer-readable medium, comprising, one or more of the following steps in any effective order, e.g., selecting a cell or gene expression profile, e.g., a profile that specifies that said gene is differentially expressed in prostate cancer, and retrieving said differentially expressed gene sequences, where the gene sequences consist of the genes represented by Table 1.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in prostate cancer. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are differentially expressed in a prostate cancer. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithm is used to interrogate the database, and retrieve results.

Advertising, Licensing, etc., Methods

The present invention also relates to methods of advertising, licensing, selling, purchasing, brokering, etc., genes, polynucleotides, specific-binding partners, antibodies, etc., of the present invention. Methods can comprises, e.g., displaying a a differentially-regulated gene gene, a differentially-regulated gene polypeptide, or antibody specific for a differentially-regulated gene in a printed or computer-readable medium (e.g., on the Web or Internet), accepting an offer to purchase said gene, polypeptide, or antibody.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found.

Reference Materials

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The preceding preferred specific embodiments are, therefore, to be construed as merely illustrative, and not limiting the remainder of the disclosure in any way whatsoever. The entire disclosure of all applications, patents and publications, cited above and in the figures are hereby incorporated by reference in their entirety.

TABLE 1

| | GI# | NM | classification | domain | description | Score | E | seq-f | seq-t |
|---|---|---|---|---|---|---|---|---|---|
| PCP0744 | 4505674 | NM_002606 | Unknown | 4505674\|ref\|NM_002606.1 Homo sapiens phosphodiesterase 9A (PDE91), mRNA | | | | | |
| 1, 2 | 4505674 | | | PDEaae | 3′5′-cyclic nucleotide phosphodiester | 225.9 | 1.30E−79 | 311 | 533 |
| | 4505674 | | | HD | HD domain | −10.5 | 0.52 | 313 | 426 |
| | 4505674 | | | gntR | Bacterial regulatory proteins, gntR f | 3.4 | 0.92 | 136 | 150 |
| PCP0729 | 11640556 | NM_014367 | Unknown | 11640556\|gb\|AF107495.1\|AF107495 Homo sapiens FWP001 and putative FWP0022 mRNA, | | | | | |
| 3, 4 | 11640556 | | | complete cds. and 6457343\|gb\|AF191020.1\|AF191020 Homo sapiens E21G5 | | | | | |
| | | | | (E21G5) mRNA, complete cds. and 9295191\|gb\|AF201944.1\|AF201944 Homo sapiens | | | | | |
| | | | | HGTD-P (HGTD-P) | | | | | |
| | | | | mRNA, complete cds. | | | | | |
| | 11640556 | | | | | | | | |
| PCP0861 | 12005804 | AF253979 | Unknown | 12005804 AF253979 Homo sapiens DC24 mRNA, complete cds | | | | | |
| 5, 6 | 12005804 | | | oxidored ql | NADH-Ubiquinone/plastiquinone | 22.9 | 9.30E−07 | 82 | 108 |
| PCP0865 | 5138913 | AF092132 | Unknown | 5138913\|gb\|AF092132.1\|AF092132 Homo sapiens PAK2 mRNA, complete cds. Also: serine | | | | | |
| | | | | kinase (hPAK65) mRNA | | | | | |
| 7, 8 | 5138913 | | | pkinase | Protein kinase domain | 284 | 9.90E−83 | 280 | 516 |
| | 5138913 | | | PBD | P21-Rho-binding domain | 120.4 | 1.80E−33 | 69 | 129 |

| PCP0731 | 10436287 | NM_022151 | Unknown | 10436287\|dbj\|AK024029.1\|AK024029 H sapiens cDNA FLJ13967 fis, clone Y79AA1001402, | | | | | |
|---|---|---|---|---|---|---|---|---|---|
| 9, 10 | 10436287 | | | weakly similar to H sapiens paraneoplastic cancer-testis-brain antigen (MA4) mRNA | | | | | |
| PCP0456 | 5209326 | NM_001634 | Unknown | 5209326\|ref\|NM_001634.3 Homo sapiens S-adenosylmethionine decarboxylase 1 (AMD1); | | | | | |
| | | | | XM_002078.3\| H. sapiens death associated protein 3 (DAP: | | | | | |
| 11, 12 | 5209326 | | | SAM decarbox | Adenosylmethionine decarboxylase | 656 | 1.00E−194 | 1 | 329 |

| PCP0660 | 10439870 | AK026903 | Unknown | 10439870\|db\|AK026903.1\|AK026903 Homo sapiens cDNA: FLJ23250 fis, clone COL0421 | | | | | |
|---|---|---|---|---|---|---|---|---|---|
| 13, 14 | 10439870 | | | Transposase 11 | Tansposase DDE domain | 139.5 | 3.20E−39 | 91 | 331 |
| PCP0389 | 11418710 | NM_014702 | Unknown | 11418710\|ref\|XM_004363.1\| Homo sapiens KIAA0408 gene product (KIAA0408), mRNA | | | | | |
| 15, 16 | 11418710 | | | | | | | | |
| PCP0590 | 12805036 | NM_024116 | Unknown | 12805036\|gbp\|BC001972.1\|BC001972 Homo sapiens, clone MGC:5306, mRNA, complete cds. | | | | | |
| 17, 18 | 12805036 | | | EST: 13580275\|gb\|BG572622.1\|BG572622 602593719F1 NIH_MGC_79 Homo sapiens cDNA | | | | | |
| | | | | clone IMAGE:4720900 5′ (plus/plus) | | | | | |
| PCP0651 | 13111807 | NM_001634 | Unknown | 13111807\|gb\|BC000171.2\|BC000171 H sapiens, S-adenosylmethionine decarboxylase 1, | | | | | |
| | | | | clone MGC:5213 mRNA, complete cds | | | | | |
| 19, 20 | 13111807 | | | SAM decarbox | Adenosylmethionine decarboxylase | 661.3 | 2.70E− | 1 | 329 |
| | | | | | | | 196 | | |
| PCP0471 | 4877277 | NM_004189 | Unknown | 4877277/AJ006230.1\|HSA6230 Homo sapiens sox-14 gene | | | | | |
| 21, 22 | 4877277 | | | HMG box | HMG (high mobility group) box | 130.1 | 2.10E−36 | 8 | 76 |
| PCP0716 | 311625 | NM_003248 | Secreted | 311625\|emb\|Z19585.1\|HSTHROMB4 H.sapiens mRNA for thromobospondin-4. EST (OriGene): | | | | | |
| | | | | BC1026 D12 E03 019.b1 (plus/minus) | | | | | |
| 23, 24 | 311625 | | | TSPN | Thrombospondin N-terminal -like domai | 205.5 | 4.20E−59 | 24 | 192 |
| | 311625 | | | tsp 3 | Thrombospondin type 3 repeat | 147.2 | 1.50E−41 | 706 | 718 |
| | 311625 | | | EGF | EGF-like domain | 37.1 | 2.10E−08 | 424 | 461 |
| | 311625 | | | ET | ET module | −33.2 | 0.14 | 353 | 430 |
| | 311625 | | | TIL | Trypsin Inhibitor like cysteine rich | −11.8 | 0.25 | 261 | 330 |
| | 311625 | | | laminin EGF | Laminin EGF-like (Domains III and V) | −14.5 | 0.47 | 289 | 324 |
| PCP0570 | 7019348 | NM_013372 | Secreted | 7019348\|ref\|NM_013372.1\| Homo sapiens cysteine knot superfamily 1, | | | | | |
| | | | | BMP antagonist 1 (CKTSF1B1), mRNA | | | | | |
| 25, 26 | 7019348 | | | DAN | DAN domain | 275.5 | 3.50E−80 | 58 | 184 |
| | 7019348 | | | TGF-beta | Transforming growth factor beta like dom | −31.5 | 0.092 | 91 | 179 |
| | 7019348 | | | HTH 6 | Helix-turn-helix domain, rpiR family | −40.3 | 0.68 | 68 | 152 |
| | 7019348 | | | PTN MK | PTN/MK heparin-binding protein family | −62.2 | 0.81 | 1 | 138 |
| PCP0624 | 1213612 | NM_006061 | Secreted | 1213612\|emb\|X94323.1\|HSSPG28 H. sapiens mRNA for SGP28 protein (specific | | | | | |
| | | | | granule protein (28 kDa); cysteine-rich secretory protein-3 (SGP28)) | | | | | |
| 27, 28 | 1213612 | | | SCP | SCP-like extracellular protein | 290.5 | 1.10E−84 | 4 | 179 |
| | 1213612 | | | Antistasin | Antistasin family | 3.3 | 0.39 | 216 | 240 |
| | 1213612 | | | TIL | Trypsin Inhibitor like cysteine rich | −17.2 | 0.72 | 184 | 238 |
| PCP0688 | 6425047 | AP188747 | Secreted | 6425047\|gb\|AF188747.1\|AF188747 Homo sapiens prostrate kallikrein 2 (KLK2) mRNA, | | | | | |
| | | | | alternatively spliced, complete cds | | | | | |
| 29, 30 | 6425047 | | | trypsin | Trypsin | 185.7 | 2.00E−59 | 25 | 209 |
| PCP0556 | 12730727 | NM_017423 | Secreted | 12730727\|ref\|XM_003527.2\| Homo sapiens UDP-N-acetyl-alpha-D-galactosamine:polypeptide | | | | | |
| 31, 32 | 12730727 | | | N-acetylgalactosaminyltransferase 7 (GaINac-T7 (GALNT7), mRNA. EST: 9793644\|gb\| | | | | | |
| | | | | BE551952.1\|BE551952 hy01f0.6x1 NCI_CGAP_GC6 Homo sapiens cDNA clone | | | | | |
| | | | | IMAGE:3196067 3′ (plus/minus) | | | | | |
| | 12730727 | | | Glycos transf 2 | Glycosyl transferase | 98.7 | 6.10E−27 | 210 | 1399 |
| | 12730727 | | | Ricin B lectin | QXW lectin repeat | 43.9 | 1.90E−10 | 617 | 652 |
| | 12730727 | | | SCP | SCP-like extracellular protein | 7.1 | 0.036 | 454 | 488 |
| PCP0497 | 13652850 | NM_001648 | Secreted | 13652850\|ref\|XM_008995.2\| Homo sapiens kallikrein 3, (prostate specific antigen) (KLK3), | | | | | |
| | | | | mRNA | | | | | |
| 33, 34 | 13652850 | | | trypsin | Trypsin | 272.6 | 5.10E−87 | 25 | 253 |
| PCP0751 | 14422306 | AJ310938 | Secreted | 14422306\|emb\|AJ310938.1\|HSA310938.1 Homo sapiens mRNA for prepro PSA-RP2 | | | | | |
| | | | | (KLK3 gene), transcript 2. (kallikrein 3, (KLK3 | | | | | |
| 35, 36 | 14422306 | | | trypsin | Trypsin | 132.6 | 1.50E−42 | 25 | 158 |
| PCP0560 | 341200 | N24543 | Secreted | 341200\|gb\|M24543.1\|HUMPSANTIG Human prostate-specific antigen (PA) gene, complete cds. | | | | | |
| 37 | 341200 | | | 35720\|emb\|X07730.1\|HSPSA Human mRNA for prostrate specific antigen (kallikrein 3) | | | | | |
| | 341200 | | | trypsin | Trypsin | 264.3 | 2.10E−84 | 23 | 254 |
| PCP0632 | 6694277 | NM_014141 | Secreted | 6694277\|gb\|AF193613.1\|AF193613 Homo sapiens cell recognition molecule Caspr2 | | | | | |
| | | | | (CASPR2) mRNA, complete | | | | | |
| 38, 39 | 6694277 | | | F5 F8 type C | F5/8 C domain | 216.5 | 2.00E−62 | 38 | 178 |
| | 6694277 | | | laminin G | Laminin G domain | 180.1 | 1.50E−51 | 1055 | 1185 |
| | 6694277 | | | EGF | EGF-like domain | 48.5 | 7.50E−12 | 967 | 1001 |
| | 6694277 | | | COLF1 | Fibrillar collagen C-terminal domain | 5.5 | 0.0062 | 611 | 632 |
| | 6694277 | | | fibrinogen C | Fibrinogen beta and gamma chains, C-t | 2.2 | 0.56 | 606 | 635 |
| | 6694277 | | | Metallothio 2 | Metallothionein | −37 | 0.96 | 690 | 745 |
| | 6694277 | | | TSPN | Thrombospondin N-terminal -like domai | −63 | 0.96 | 352 | 528 |
| PCP0402 | 13529043 | NM_001648 | Secreted | 13529043\|gb\|BC005307.1\|BC005307 Homo sapiens, kallikrein 3, (prostate specific antigen), clone | | | | | |
| 40, 41 | 13529043 | | | trypsin | Trypsin | 272.6 | 5.10E−87 | 25 | 253 |
| PCP0421 | 13653145 | NM_013372 | Secreted | 13653145\|ref\|XM_007593.3\| Homo sapiens cysteine knot superfamily 1, BMP antagnoist 1 | | | | | |
| 42, 43 | 13653145 | | | DAN | DAN domain | 243.3 | 1.70E−70 | 1 | 117 |
| | 13653145 | | | TGF-beta | Transforming growth factor beta like dom | −31.5 | 0.092 | 24 | 112 |
| | 13653145 | | | HTH 6 | Helix-turn-helix domain, rpiR family | −40.3 | 0.68 | 1 | 85 |
| PCP0576 | 13643135 | NM_006061 | Secreted | 13643135\|ref\|XM_004475.3\| Homo sapiens specific granule protein (28 kDa): | | | | | |
| | | | | cystein-rich secretory protein-3 (SGP28), mRNA | | | | | |
| 44, 45 | 13643135 | | | SCP | SCP-like extracellular protein | 287.1 | 1.10E−83 | 4 | 179 |
| | 13643135 | | | Antistasin | Antistasin family | 3.3 | 0.39 | 216 | 240 |
| | 13643135 | | | TIL | Trypsin Inhibitor like cysteine rich | −17.2 | 0.72 | 184 | 238 |
| PCP0486 | 1770465 | XM_167894 | Nuclear | 1770465\|emb\|X98259.1\|HSMPP8 H.sapiens mRNA for M-phase phosphoprotein, mpp8 | | | | | |
| 46, 47 | 1770465 | | | | | | | | |
| PCP0858 | 35569 | NM_002568 | Nuclear | 35569 Y00345.1\|HSPOLYAB Human mRNA for polyA binding protein | | | | | |
| 48, 49 | 35569 | | | rrm | RNA recognition motif (a.k.a. RRM,R | 373.3 | 1.30E− | 293 | 362 |
| | | | | | | | 109 | | |
| | 35569 | | | PABP | Poly-adenylate binding protein, uniqu | 168.6 | 5.30E−48 | 540 | 611 |
| PCP0404 | 298110 | NM_000123 | Nuclear | 298110\|emb\|X69978.1\|HSXPGAA H.sapiens mRNA for XP-G factor | | | | | |
| 50, 51 | 298110 | | | XPG-N | XPG N-terminal domain | 197.7 | 9.30E−57 | 1 | 98 |
| | 298110 | | | XPG I | XPG I-region | 167.1 | 1.60E−47 | 777 | 865 |
| | 298110 | | | DNA po13 beta | DNA polymerase III beta subunit, N-tc | 6.5 | 0.12 | 383 | 403 |
| | 298110 | | | Ribosomal S21 | Ribosomal protein S21 | −17.7 | 0.38 | 1038 | 1082 |
| PCP0457 | 435422 | NM_002015 | Nuclear | 435422\|gb\|U02310.1\|HSU02310 Human fork head domain protein (FKHR) mRNA, complete cds | | | | | |
| 52, 53 | 435422 | | | Fork head | Fork head domain | 87.8 | 1.10E−23 | 122 | 242 |
| | 435422 | | | X | Trans-activation protein X | 1.3 | 0.41 | 56 | 76 |
| | 435422 | | | 60s ribosomal | 60s Acidic ribosomal protein | 1.5 | 0.91 | 76 | 102 |
| PCP0859 | 560622 | NM_001788 | Nuclear | 560622 S72008 hCDC10=DC10 homolog [human, fetal lung, mRNA, 2314 nt] | | | | | |
| 54, 55 | 560622 | | | GTP CDC | Cell division protein | 589.9 | 8.10E− | 28 | 304 |
| | | | | | | | 175 | | |
| | 560622 | | | M | M protein | 15.2 | 0.083 | 366 | 386 |
| | 560622 | | | Ribosomal L36 | Ribosomal protein L36 | 5 | 0.13 | 344 | 380 |
| | 560622 | | | STAT | STAT protein, all-alpha domain | −65.7 | 0.21 | 256 | 401 |
| | 560622 | | | Arginosuc synth | Arginosuccinate synthase | 0.7 | 0.65 | 5 | 25 |
| | 560622 | | | ATP-bind | Conserved hypothetical ATP binding pr | −135.9 | 0.69 | 33 | 229 |
| | 560622 | | | ras | Ras family | −142 | 0.77 | 33 | 187 |
| PCP0654 | 4008100 | AF107043 | Nuclear | 4008100\|gb\|AF107043.1\|AF107043 Homo sapiens clone pCL11 DNA-binding protein SOX14 | | | | | |
| | | | | (SOX14) gene, complete cds | | | | | |
| 56, 57 | 4008100 | | | HMG box | HMG (high mobility group) box | 130.1 | 2.10E−36 | 8 | 76 |
| PCP0514 | 4504444 | NM_002136 | Nuclear | 4504444\|ref\|NM_002136.1\| Homo sapiens heterogeneous nuclear ribonucleoprotein A1 | | | | | |
| 58, 59 | 4504444 | | | (HNRPA1), mRNA (and gb\|U00947.1\|U00947 Human clone C4E 3.2 (CAC)n/(GTG)n | | | | | |
| | | | | repeat-containing mRNA) | | | | | |
| | 4504444 | | | rrm | RNA recognition motif. (a.k.a. RRM, R | 178.9 | 4.20E−51 | 107 | 177 |
| PCP0679 | 4557448 | NM_001271 | Nuclear | 4557448\|ref\|NM_001271.1 Homo sapiens chromodomain helicase DNA binding proteint 2 | | | | | |
| | | | | (CHD2), mRNA | | | | | |
| 60, 61 | 4557448 | | | SNF2 N | SNF2 and others N-terminal domain | 474 | 6.40E− | 487 | 768 |
| | | | | | | | 140 | | |
| | 4557448 | | | chromo | ‘chromo’ (CHRromatin Organization | 98.5 | 9.30E−29 | 375 | 433 |
| | | | | | MOd | | | | |
| | 4557448 | | | helicase C | Helicase conserved C-terminal domain | 103 | 3.00E−28 | 821 | 905 |
| | 4557448 | | | myb DNA- | Myb-like DNA-binding domain | 14.9 | 0.00078 | 1258 | 1284 |
| | | | | binding | | | | | |
| | 4557448 | | | AT hook | AT hook motif | 9.3 | 0.22 | 1114 | 3126 |
| | 4557448 | | | TP2 | Nuclear trasnition protein 2 | −58.5 | 0.35 | 2 | 113 |
| | 4557448 | | | Ribosomal L36 | Ribosomal protein L36 | 2.4 | 0.96 | 752 | 769 |
| PCP0485 | 9963969 | NM_006167 | Nuclear | 9963969\|gb\|AF247704.1\|AF247704 Homo sapiens homeobox protein NKX3.1 mRNA, | | | | | |
| | | | | complete cds | | | | | |
| 62, 63 | 9963969 | | | homeobox | Homeobox domain | 101 | 1.20E−27 | 125 | 181 |
| PCP0618 | 13623684 | BC006470 | Nuclear | 13623684\|gb\|BC006470.1\|BC006470 Homo sapiens, Similar to macrophase erythroblast, | | | | | |
| | | | | attacher, clone MGC:3775 IMAGE:2823478, mRNA, complete cds | | | | | |
| 64, 65 | 13623684 | | | Ribosomal L22 | Ribosomal protein L22p/L17e | 7.5 | 0.04 | 29 | 4 |
| | 13623684 | | | Luteo ORF3 | Luteovirus (ORF3) RNA-directed RNA-p | 1.4 | 0.38 | 34 | 60 |
| PCP0419 | 13655140 | NM_001675 | Nuclear | 1365514\|ref\|XM_010004.2\| Homo sapiens activating factor 4 (tax-responsive ehance element | | | | | |
| | | | | B67) (ATF4), mRNA | | | | | |
| 66, 67 | 13655140 | | | bZIP | bZIP transcription factor | 73.3 | 1.50E−20 | 276 | 340 |
| | 13655140 | | | K-box | K-box region | −45.6 | 0.83 | 253 | 333 |
| PCP0673 | 14721378 | NM_014367 | Membrane | 14721378\|ref\|XM_002805.4 Homo sapiens hypothetical protein, estradiol-induced | | | | | |
| 68, 69 | 14721378 | | | (E21KG5), mRNA | | | | | |
| PCP0827 | 190663 | NM_004476 | Membrane | 190663 M99487.1\|HUMPSM Human prostate-specific membrane antigen (PSM) mRNA, | | | | | |
| | | | | complete cds | | | | | |
| 70, 71 | 190663 | | | PA | PA domain | 99.2 | 4.20E−27 | 165 | 265 |
| | 190663 | | | MutS C | DNA mismatch repair proteins mutS fa | 2.2 | 0.77 | 110 | 118 |
| PCP0504 | 182904 | M93036 | Membrane | 182904\|Gb\|M93036.1\|HUMGA7A08 Human (clone 21726) carcinoma-associatod antigen | | | | | |
| 72, 73 | 182904 | | | GA733-2 (GA733-2) mRNA, exon 9 and complete cds, 10439469\|dbj\|AK026585.1\| | | | | | |
| | | | | AK026585 Homo sapiens cDNA: FLJ22932 fis, cone KAT07515, highly similiar to HUMGA7A | | | | | |
| | | | | Human (clone GA733-2-2) | | | | | |
| | 182904 | | | thryoglobulin 1 | Thyroglobulin type-1 repeat | 104.5 | 1.10E−28 | 66 | 135 |
| PCP0508 | 2352837 | AF009255 | Membrane | 2352837\|gb\|AF009255.1\|CLCN6HUM09 Homo sapiens putative chloride channel CLCN6, | | | | | |
| | | | | exons 14, 15, 16, and 17 | | | | | |
| 74, 75 | 2352837 | | | voltage CLC | Voltage gated chloride channel | 649.1 | 1.30E− | 136 | 572 |
| | | | | | | | 192 | | |
| | 2352837 | | | CBS | CBS domain | 95.4 | 5.80E−26 | 805 | 858 |
| | 2352837 | | | K trans | K+ potassium transporter | −566.6 | 0.3 | 313 | 803 |
| PCP0605 | 12654454 | NM_001307 | Membrane | 12654454\|gb\|BC00105.1\|BC001055 Homo sapiens, claudin 7, clone MGC:1858, mRNA, | | | | | |
| 76, 77 | 12654454 | | | complete cdsEST: 14058040\|gb\|BG747387.1\|BG747387 602704864F1 NIH_MGC_15 | | | | | |
| | | | | Homo sapiens cDNA clone IMAGE:4858239 5′ (plus/plus) | | | | | |
| | 12654454 | | | PMP22 Claudin | PMP-22/EMP/MP20/Claudin family | 338.4 | 4.20E−99 | 4 | 182 |
| PCP0713 | 183990 | NM_001982 | Membrane | 183990\|gb\|M34309.1\|HUMHER3A Human epidermal growth factor receptor (HER3) mRNA, | | | | | |
| 78, 79 | | | | complete cds. Also: avian erythroblastic leukemia viral oncogene homolog 3 (ERBB3). | | | | | |
| | 183990 | | | Recep L domain | Receptor L domain | 413.6 | 9.40E− | 364 | 486 |
| | | | | | | | 122 | | |
| | 183990 | | | Furin-like | Furin-like cysteine rich region | 337.3 | 8.70E−99 | 180 | 332 |
| | 183990 | | | pkinase | Protein kinase domain | 216.8 | 1.70E−62 | 709 | 961 |
| | 183990 | | | Keratin B2 | Keratin, high sulfur B2 protein | −79.1 | 0.26 | 180 | 323 |
| | 183990 | | | fer4 | 4Fe-4S binding domain | −3.1 | 0.41 | 278 | 296 |
| | 183990 | | | TNFR c6 | TNFR/NGFR cysteine-rich region | −3.2 | 0.92 | 539 | 576 |
| PCP0852 | 4557340 | NM_001183 | Intracellular | 4557340 NM_001183.1\| Homo sapiens ATPase H+ transporting, lysosomal (vacular | | | | | |
| | | | N) | | | | | | |
| 80, 81 | | | | proton pump) | | | | | |
| PCP0792 | 7862074 | AF245699 | Membrane | 7862074\|gb\|AF45699.1\|AF245699 Homo sapiens type 1 angiotensin II receptor (AGTR1) gene, | | | | | |
| | | | | complete cds | | | | | |
| 82, 83 | 7862074 | | | 7tm 1 | 7 transmembrane receptor (rhodopsin f | 328.5 | 5.20E− | 45 | 302 |
| | | | | | | | 104 | | |
| PCP0627 | 13365514 | NM_130386 | Membrane | 13365514\|dbj\|AB038518.1\|AB038518 Homo sapiens SRCL mRNA for scavenger receptor | | | | | |
| | | | | with C-type lectin type | | | | | |
| 84, 85 | 13365514 | | | lectin c | Lectin C-type domain | 126.5 | 2.60E−35 | 624 | 732 |
| | 13365514 | | | Collagen | Collagen triple helix repeat (20 copi | 100.3 | 2.00E−27 | 527 | 586 |
| | 13365514 | | | Syntaxin | Syntaxin | −102.3 | 0.25 | 65 | 353 |
| | 13365514 | | | RNA pol | DNA-dependent RNA polymerase | −245.3 | 0.28 | 128 | 646 |
| | 13365514 | | | Arginosuc synth | Arginosuccinate synthase | 1.3 | 0.42 | 711 | 729 |
| | 13365514 | | | beta-lactamase | Beta-lactamase | 3 | 0.42 | 668 | 698 |
| | 13365514 | | | DUF164 | Uncharacterized ACR, COG1579 | −99.5 | 0.54 | 244 | 444 |
| | 13365514 | | | ERM | Ezrin/radixin/moesin family | −226.8 | 0.61 | 80 | 405 |
| | 13365514 | | | FAT | FAT domain | −189.1 | 0.9 | 8 | 673 |
| | 13365514 | | | HrcA | HrcA protein C terminal domain | −107.8 | 0.92 | 167 | 360 |
| PCP0735 | 13543530 | NM_020300 | Membrane | 13543530\|gb\|BC005923.1\|BC005923 Homo sapiens, microsomal glutathione S-transferase 1, | | | | | |
| | | | | clone MGC:14525, mRNA, complete cds | | | | | |
| 86, 87 | 13543530 | | | MAPEG | MAPEG family | 163.9 | 1.40E−46 | 67 | 155 |
| | 13543530 | | | SSB | Single-strand binding protein family | 3.3 | 0.66 | 49 | 64 |
| PCP0680 | 183684 | NM_006931 | Membrane | 183684\|gb\|M20681.1\|HUMGTLPA Human glucose transporter-like protein-III (GLUT3), | | | | | |
| | | | | complete cds | | | | | |
| 88, 89 | 183684 | | | sugar tr | Sugar (and other) transporter | 663.9 | 4.30E− | 12 | 465 |
| | | | | | | | 197 | | |
| | 183684 | | | casein kappa | Kappa casein | 39 | 0.26 | 91 | 103 |
| | 183684 | | | ion trans | Ion transport protein | −14.9 | 0.69 | 12 | 145 |
| | 183684 | | | GntP permease | GntP family permease | −385.8 | 0.72 | 108 | 448 |
| | 183684 | | | 7tm 3 | 7 transmembrane receptor (metabotropi | −165.2 | 0.93 | 299 | 491 |
| | 183684 | | | Na H Exchanger | Sodium/hydrogen exchanger family | −191.6 | 0.95 | 69 | 395 |
| PCP0479 | 14781632 | NM_003144 | Membrane | 14781632\|ref\|XM_004462.4 Homo sapiens signal sequence receptor, alpha (translocon- | | | | | |
| | | | | associated protein alpha) (SSR1), mRNA | | | | | |
| 90, 91 | 14781632 | | | PAC | PAC motif | 7 | 0.14 | 17 | 38 |
| PCP0854 | 7108342 | NM_004417 | Intracellular | 7108342 NM_004417.2\| Homo sapiens dual specificity phosphatase 1 (DUSP1), mRNA | | | | | |
| 92, 93 | 7108342 | | | DSPc | Dual specificity phosphatase, catalytic | 263.4 | 1.60E−76 | 173 | 311 |
| | 7108342 | | | Rhodanese | Rhodanese-like domain | 94 | 1.50E−25 | 9 | 131 |
| | 7108342 | | | Y phosphatase | Protein-tyrosine phosphatase | −9.5 | 0.09 | 146 | 308 |
| PCP0749 | 14149664 | NM_015070 | Intracellular | 14149664\|ref\|NM_015070.1 Homo sapiens KIAA0853 protein (KIAA0853), mRNA | | | | | |
| 94, 95 | 14149664 | | | fer4 NifH | 4fe-4s iron sulfur cluster binding pr | 6.1 | 0.047 | 1069 | 1087 |
| PCP0705 | 854166 | NM_003768 | Intracellular | 854166\|emb\|X86809.1\|HPEA15 Homo sapiens mRNA for major astrocytic phosphoprotein | | | | | |
| | | | | PEA-15 | | | | | |
| 96, 97 | 854166 | | | DED | Death effector domain | 122.5 | 4.10E−34 | 3 | 87 |
| PCP0748 | 7669552 | NM_007126 | Intracellular | 7669552\|ref\|NM_007176.2 Homo sapiens valosin-containing protein (VCP), mRNA. | | | | | |
| | | | | Also: transitional endoplasmic reticulum ATPase mRNA | | | | | |
| 98, 99 | 7669552 | | | AAA | ATPase family associated with various | 653.2 | 6.90E− | 513 | 700 |
| | | | | | | | 194 | | |
| | 7669552 | | | cdc48 N | Cell division protein 48 (CDC48), N-t | 142.2 | 4.80E−40 | 22 | 108 |
| | 7669552 | | | Sigma54 actival | Sigma-54 interaction domain | 11.5 | 0.0012 | 514 | 531 |
| | 7669552 | | | NB-ARC | NB-ARC domain | 9.5 | 0.0036 | 516 | 534 |
| | 7669352 | | | Viral helicase1 | Viral (Superfamily 1) RNA helicase | 7.1 | 0.017 | 514 | 578 |
| | 7669552 | | | Exonuc VII S | Exonuclease VII small subunit | 5.6 | 0.087 | 394 | 442 |
| | 7669552 | | | bac dnaA | Bacterial dnaA protein | 2.3 | 0.22 | 514 | 539 |
| | 7669552 | | | ABC tran | ABC transporter | −67.7 | 0.25 | 511 | 700 |
| | 7669552 | | | SRP54 | SRP54-type protein, GTPase domain | 3.7 | 0.3 | 236 | 252 |
| | 7669552 | | | SKI | Skikimate kinase | −78 | 0.32 | 239 | 389 |
| | 7669552 | | | Adeno IVa2 | Adenovirus IVa2 protein | −251.1 | 0.36 | 437 | 712 |
| | 7669552 | | | adenylatekinase | Andenylate kinase | 3.6 | 0.38 | 516 | 524 |
| | 7669552 | | | 2-oxoacid dh | 2-oxo acid dehydrogenases acyltransfe | −158.8 | 0.47 | 279 | 464 |
| | 7669552 | | | RNA helicase | RNA helicase | −138.7 | 0.55 | 409 | 727 |
| | 7669352 | | | UPF0079 | Uncharacterised P-Loop hydrolase UPF0 | −63.7 | 0.6 | 219 | 336 |
| | 7669552 | | | Transposase 9 | Transposase | −20.4 | 0.82 | 411 | 502 |
| PCP0420 | 13636423 | NM_001615 | Intracellular | 13636423\|ref\|XM_010801.2\|Homo sapiens actin, gamma 2, smooth muscle, enteric (ACTG2), | | | | | |
| | | | | mRNA | | | | | |
| 100, 101 | 13636423 | | | actin | Actin | 929.1 | 5.00E− | 1 | 376 |
| | | | | | | | 284 | | |
| PCP0482 | 13124878 | NM_002474 | Intracellular | 13124878\|ref\|NM_002474.1\| Homo sapiens myosin, heavy polypeptide 11, smooth muscle | | | | | |
| 102, 103 | 13124878 | | | (MYH11), transcript variant SM1, mRNA. EST: 1437234\|gb\|BG954178.1\| | | | | | |
| | | | | BG954178 RC4-CT0628-060201-032-h09 CT0628 Homo sapiens cDNA (plus/plus) | | | | | |
| | 13124878 | | | myosin head | Myosin head (motor domain) | 1528.4 | 0 | 87 | 771 |
| | 13124878 | | | Myosin tail | Myosin tail | 778.1 | 1.80E− | 1073 | 1931 |
| | | | | | | | 231 | | |
| | 13124878 | | | M | M protein repeat | 89.9 | 2.60E−24 | 1877 | 1897 |
| | 13124878 | | | Myosin N | Myosin N-terminal SH3-like domain | 75.8 | 4.70E−20 | 33 | 77 |
| | 13124878 | | | IQ | IQ calmodulin-biding motif | 22 | 0.00076 | 787 | 807 |
| | 13124878 | | | bZIP | bZIP transcription factor | 13.4 | 0.0015 | 1788 | 1817 |
| | 13124878 | | | DUF164 | Uncharacterized ACR, COG1579 | −84.3 | 0.063 | 1018 | 1240 |
| | 13124878 | | | Lipoprotein 1 | Borrelia lipoprotein | 4 | 0.075 | 1779 | 1809 |
| | 13124878 | | | Troponin | Troponin | −23.5 | 0.12 | 1199 | 1346 |
| | 13124878 | | | Syntaxin | Syntaxin | −97.5 | 0.12 | 1609 | 1866 |
| | 13124878 | | | filament | Intermediate filament protein | 4.7 | 0.16 | 1072 | 1105 |
| | 13124878 | | | K-box | K-box region | −37.9 | 0.19 | 964 | 1062 |
| | 13124878 | | | HR1 | Hr1 repeat motif | −0.9 | 0.23 | 1034 | 1115 |
| | 13124878 | | | SMC C | SMC family C-terminal domain | −130.2 | 0.28 | 1190 | 1323 |
| | 13124878 | | | spectrin | Spectrin repeat | 5.2 | 0.29 | 1647 | 1677 |
| | 13124878 | | | DUF232 | Putative transcriptional regulator | −32.9 | 0.29 | 1303 | 1436 |
| | 13124878 | | | UBX | UBX domain | −12.9 | 0.33 | 1256 | 1327 |
| | 13124878 | | | PstS | Phosphate-binding protein | 2.5 | 0.45 | 311 | 325 |
| | 13124878 | | | Tropomyosin | Tropomyosin | 1.6 | 0.57 | 1680 | 1701 |
| | 13124878 | | | VanY | D-alanyl-D-alanine carboxypeptidase | −76.5 | 0.61 | 152 | 275 |
| | 13124878 | | | Apolipoprotein | Apolipoprotein A1/A4/E family | −121.2 | 0.67 | 1484 | 1722 |
| | 13124878 | | | Phosphoprotein | Vesiculovirus phosphoprotein | 0.2 | 0.67 | 1087 | 1101 |
| | 13124878 | | | BAR | BAR domain | −100 | 0.72 | 1325 | 1534 |
| | 13124878 | | | Borrelia orfA | Borrelia ORF-A | −107.1 | 0.72 | 1194 | 1512 |
| | 13124878 | | | SOR SNZ | SOR/SNZ family | −42.3 | 0.76 | 1148 | 1351 |
| | 13124878 | | | OEP | Outer membrane efflux protein | −45.8 | 0.79 | 1548 | 1733 |
| | 13124878 | | | Involucrin | Involucrin repeat | 8.8 | 0.88 | 1844 | 1853 |
| | 13124878 | | | hexokinase | Hexokinase | 1.1 | 0.91 | 1370 | 1403 |
| | 13124878 | | | Exo70 | Exo70 exocyst complex subunit | −284 | 0.93 | 1010 | 1546 |
| | 13124878 | | | Transposase 12 | Transposase | −163.8 | 0.95 | 1257 | 1558 |
| | 13124878 | | | B56 | Protein phosphatase 2A regulatory B₈ | 0.6 | 0.96 | 1402 | 1434 |
| PCP0780 | 14250064 | NM_001743 | Intracellular | 14250064\|gb\|BC008437.1\|BC008437 Homo sapiens, calmodulin 2 (phosphorylase kinase, | | | | | |
| | | | | delta), clone MGC:14639, mRNA, complete cds | | | | | |
| 104, 105 | 14250064 | | | efhand | EF hand | 168.8 | 4.80E−48 | 121 | 149 |
| | 14250064 | | | RmaAD | Ribosomal RNA adenine dimethylase | 6.5 | 0.016 | 111 | 147 |

| | | | | |
|---|---|---|---|---|
| PCP0807 | 13111800 | XM_167414 | Intracellular | 13111800\|gb\|BC000163.2\|BC000163 Homo sapiens, vimentin, clone MGC:5062, mRNA, |
| | | | | complete cds |

| | | | | | | | |
|---|---|---|---|---|---|---|---|
| 106, 107 | 13111800 | filament | Intermediate filament protein | 593.6 | 6.30E− | 102 | 410 |
| | | | | | 176 | | |
| | 13111800 | bZIP | bZIP transcription factor | 7.8 | 0.057 | 354 | 378 |
| | 13111800 | Apolipoprotein | Apolipoprotein A1/A4/E family | −101.5 | 0.089 | 85 | 366 |
| | 13111800 | Rota NS26 | Rotavirus NS26 | −106.1 | 0.18 | 1 | 168 |
| | 13111800 | DUF232 | Putative transcriptional regulator | −29.9 | 0.19 | 192 | 330 |
| | 13111800 | DUF164 | Uncharacterized ACR, COG1579 | −94.7 | 0.27 | 178 | 408 |

| PCP0392 | 13655369 | NM_004010 | Intercellular | 13655369\|ref\|XM_017106.1\| Homo sapiens dystrophin (muscular dystrophy, Duchenne | | | | | |
|---|---|---|---|---|---|---|---|---|---|
| | | | | and Becker | | | | | |
| 108, 109 | 13655369 | | | spectrin | Spectrin repeat | 501.6 | 3.00E− | 1556 | 1657 |
| | | | | | | | 148 | | |
| | 13655369 | | | CH | Calponin homology (CH) domain | 134 | 1.40B.37 | 11 | 117 |
| | 13655369 | | | Apolipoprotein | Aplipoprotein A1/A4/E family | −103.8 | 0.11 | 785 | 1051 |
| | 13655369 | | | KE2 | KE2 family protein | −41.9 | 0.15 | 1360 | 1467 |
| | 13655369 | | | DUF164 | Uncharacterized ACR, COG1579 | −98.5 | 0.47 | 907 | 1111 |
| | 13655369 | | | SPX | SPX domain | −76.1 | 0.53 | 865 | 981 |
| | 13655369 | | | pou | Pou domain-N-terminal to homeobox d | 4 | 0.54 | 1156 | 1163 |
| | 13655369 | | | MuDR | MuDR family transposase | −60.7 | 0.66 | 462 | 618 |
| | 13655369 | | | OspEF | Borrelia outer surface protein E and | −84.5 | 0.73 | 1131 | 1309 |
| | 13655369 | | | FliD | Flagellar hook-associated protein 2 | −251.9 | 0.89 | 466 | 943 |
| | 13655369 | | | DUF118 | Helix-turn-helix family DUF118 | −83.5 | 0.92 | 1147 | 1361 |
| PCP0808 | 14734368 | NM_000944 | Cytoplasm | 14734368\|ref\|XM_003316.3 Homo sapiens protein phosphatase 3 (formerly 2B), catalytic | | | | | |
| 110, 111 | 14734368 | | | subunit, alpha isoform (calcineurin A alpha) (PPP3CA), mRNA. 3 end of the DD sequence | | | | | |
| | | | | matches genomic DNA in the intron region | | | | | |
| | 14734368 | | | Stphosphatase | Ser/Thr protein phosphatase | 479.3 | 1.10E− | 43 | 339 |
| | | | | | | | 141 | | |
| PCP0511 | 11427423 | NM_004152 | Cytoplasm | 11427423\|ref\|XM_009414.1\| Homo sapiens ornithine decarboxylase antizyme 1 (OAZ1), | | | | | |
| 112, 113 | | | | mRNA. EST:11642610\|gb\|BF569319.1\|BF569319 602184675T1 NIH_MGC_42 | | | | | |
| | | | | Homo sapiens cNDA clone IMAGE:4300434 3′(plus/minus) | | | | | |
| | 11427423 | | | ODC AZ | Ornithine decarboxylase antizyme | 129.1 | 3.50E−38 | 2 | 68 |
| PCP0647 | 13647403 | NM_001182 | Cytoplasm | 13647403\|ref\|XM_017407.1\| Homo sapiens similar to aldehyde dehydrogenase 7 family, | | | | | |
| | | | | member A1 | | | | | |
| 114, 115 | 13647403 | | | aldedh | Aldehyde dehydrogenase family | 122.0 | 3.00E−34 | 1 | 318 |
| PCP0502 | 14149943 | NM_032236 | Cytoplasm | 14149943\|ref\|NM_032236.1 Homo sapiens hypothetical protein FLJ23277 (FLJ23277), mRNA | | | | | |
| 116, 117 | 14149943 | | | UCH-1 | Ubiquitin carboxyl-terminal hydrolase | 31.6 | 4.50E−07 | 89 | 120 |
| | 14149943 | | | UCH-2 | Ubiquitin carboxyl-terminal hydrolase | 14.6 | 3.10E−05 | 332 | 377 |
| PCP0823 | 1373418 | NM_001000 | Cytoplasm | 1373418\|gb\|U57846.1\|HSU57846 Human ribosomal protein L39 mRNA, complete cds | | | | | |
| 118, 119 | 1373418 | | | Ribosomal L39 | Ribosomal L39 protein | 100.6 | 1.60E−27 | 9 | 51 |
| PCP0598 | 12804630 | NM_016548 | Cytoplasm | 12804630\|gb\|BC001740.1\|BC001740 Homo sapiens, golgi membrane protein GP73, clone | | | | | |
| 120, 121 | | | | MGC:850, mRNAEST: 10939527\|gb\|BF109837.1\|BF109837 7169h08.x1 Saores NSF F8 9W OT | | | | | |
| | | | | PA P S1 Homo sapiens cDNA clone (plus/minus) | | | | | |
| | 12804630 | | | Phosphoprotein | Vesiculovirus phosphoprotein | −0.2 | 0.91 | 121 | 158 |
| PCP9657 | 13529097 | NM_000990 | Cytoplasm | 13529097\|gb\|BC005326.1\|BC005326 Homo sapiens, ribosomal protein L27a, clone | | | | | |
| | | | | MGC:12412, mRNA, complete cds | | | | | |
| 122, 123 | 13529097 | | | L15 | Ribosomal protein L15 | 61.9 | 1.80E−22 | 110 | 142 |
| | 13529097 | | | Arena glycoprol | Arenavirus glycoprotein | −1.1 | 0.91 | 107 | 114 |
| PCP0559 | 13637676 | NM_002539 | Cytoplasm | 13637676\|ref\|XM_002679.2\| Homo sapiens ornithine decarboxylase 1 (ODC1), mRNAEST: | | | | | |
| 124, 125 | 13637676 | | | 12936623\|emb\|AL575449.1\|AL575449 AL575449 LTI NFL006 PL2 Homo sapiens cDNA clone | | | | | |
| | | | | CS0DI060YF19 3 (plus/minus) | | | | | |
| | 13637676 | | | Orn Arg deC N | Pyridoxal-dependent decarboxylase, py | 326 | 7.40E−97 | 44 | 222 |
| PCP0650 | 13642887 | NM_001634 | Cytoplasm | 13642887\|ref\|XM_011385.2\| Homo sapiens S-adenosylmethionine decarboxylase 1 (AMD1), | | | | | |
| | | | | mRNA | | | | | |
| 126, 127 | 13642887 | | | SAM decarbox | Adenosylmethionine decarboxylase | 661.3 | 2.70E− | 1 | 329 |
| | | | | | | | 196 | | |
| PCP0830 | 14044036 | NM_001015 | Cytoplasm | 14044036\|bg\|BC007945.1\|BC007945 Homo sapiens, ribosomal protein S11, clone | | | | | |
| | | | | MGC:143222 IMAGE:4297932, mRNA, complete cds. | | | | | |
| 128, 129 | 14044036 | | | Ribosomal S17 | Ribosomal protein S17 | 149.1 | 2.00E−44 | 75 | 145 |
| PCP0760 | 14195600 | NM_032105 | Cytoplasm | 14195600\|ref\|NM_032105.1 Homo sapiens myosin phosphatase, taget subunit 2 (MYPT2), | | | | | |
| | | | | transcript variant 2, mRNA | | | | | |
| 130, 131 | 14195600 | | | ank | Ank repeat | 177.4 | 1.20E−50 | 249 | 281 |
| | 14195600 | | | G-gamma | GGL domain | 4.1 | 0.34 | 962 | 975 |
| | 14195600 | | | bZIP | bZIP transcription factor | 4.3 | 0.58 | 942 | 978 |
| | 14195600 | | | RNA pol | DNA dependent RNA polymerase | −250.8 | 0.6 | 74 | 552 |
| | 14195600 | | | Adaptin N | Adaptin N terminal region | 1.2 | 0.71 | 600 | 627 |
| PCP0555 | 14198285 | NM_014320 | Cytoplasm | 14198285\|gb\|BC008205.1\|BC008205 Homo sapiens, putative heme-binding protein, clone | | | | | |
| 132, 133 | 14198285 | | | MGC:17713, mRNA, complete cdsEST: 13793234\|gb\|BG65582.1\|BG655825 ib40e02.x1 | | | | | |
| | | | | HR85 islet Homo sapiens cDNA 3′ similar to TR:Q9Y5Z4 (plus/minus) | | | | | |
| | 14198285 | | | | | | | | |
| PCP0799 | 14603308 | BC010112 | Cytoplasm | 14603308\|gb\|BC010112.1\|BC010112 Homo sapiens, heat shock 60kD protein 1 (chaperonin), | | | | | |
| | | | | clone MGC:19755, mRNA, complete cds | | | | | |
| 134, 135 | 14603308 | | | cpn60 TCP1 | TcP-1/cpn60 chaperonin family | 723.2 | 6.20E− | 37 | 540 |
| | | | | | | | 215 | | |
| | 14603308 | | | Asparaginase | Asparaginase | −166.5 | 0.79 | 69 | 359 |
| | 14603308 | | | NB-ARC | NB-ARC domain | 1.3 | 0.88 | 353 | 394 |
| PCP0468 | 179829 | NM_004342 | Cytoplasm | 179829\|gb\|M64110.1\|HUMCALD Human caldesmon mRNA, complete cds | | | | | |
| 136,137 | 179829 | | | Caldesmon | Caldesmon | 1057.2 | 0 | 1 | 538 |
| | 179829 | | | G-gamma | GGL domain | 4.5 | 0.24 | 346 | 367 |
| PCP0546 | 338234 | M33216 | Cytoplasm | 338234\|gb\|M33216.1\|HUMSMAAA Human aortic-type smooth muscle alpha-actin | | | | | |
| 138, 139 | 338235 | | | (SM-alpha-A) gene, exon 9. EST: 13581056\|gb\|BG573403.1\|BG573403 602595180F | | | | | |
| | | | | NIH_MGC_79 Homo sapiens cDNA clone IMAGE:4724587 ′ (plus/minus) | | | | | |
| | 338234 | | | actin | Actin | 112.5 | 3.50E−35 | 1 | 47 |
| PCP0667 | 809558 | NM_000256 | Cytoplam | 809558\|emb\|X84075.1\|HSMYBPC H.sapiens mRNA for cardiac myosin binding protein-C | | | | | |
| 140, 141 | 809558 | | | fn3 | Fibronectin type III domain | 170.5 | 1.40E−48 | 1066 | 1151 |
| | 809558 | | | ig | Immunoglobulin domain | 127.5 | 2.80E−39 | 1195 | 1255 |
| | 809558 | | | ATP-gus Ptrans | ATP:guanldo phosphotransferase, C-ter | 4.7 | 0.095 | 536 | 559 |
| | 809558 | | | Bima VP3 | Bimavirus VP3 protein | 2 | 0.62 | 827 | 839 |
| PCP0488 | 1143491 | NM_005347 | Cytoplasm | 1143491\|emb\|X87949.1\|HSRNABIP H.sapiens mRNA for BiP protein | | | | | |
| 142, 143 | 1143491 | | | HSP70 | Hsp70 protein | 1415.1 | 0 | 130 | 635 |
| | 1143491 | | | Hydantoinase A | Hydantoinase/oxoprolinase | −330.3 | 0.086 | 30 | 371 |
| | 1143491 | | | Ppx-GppA | Ppx/GppA phosphatase family | −136.9 | 0.8 | 115 | 362 |
| | 1143491 | | | Fibrillarin | Fibrillarin | −128.7 | 0.85 | 150 | 366 |
| PCP0656 | 2370089 | NM_000366 | Cytoplasm | 2370089\|emb\|AJ000147.1\|HSAJ147 Homo sapiens mRNA for alpha-tropmyosin (3′ end) | | | | | |
| 144, 145 | 2370089 | | | Tropomyosin | Tropomyosin | 97.1 | 1.30E−31 | 2 | 48 |
| PCP0707 | 3327967 | NM_002606 | Cytoplasm | 3327967\|gb\|AF067223.1\|AF067223 Homo sapiens cGMP phosphodiestarase A1 (PDE9A) | | | | | |
| | | | | mRNA, complete cds | | | | | |
| 146, 147 | 3327967 | | | PDEase | 3′5′-cyclic nucleotide phosphodiester | 225.9 | 1.30E−79 | 311 | 533 |
| | 3327967 | | | HD | HD domain | −10.5 | 0.52 | 313 | 426 |
| | 3327967 | | | gntR | Bacterial regulator proteins, gntR f | 3.4 | 0.92 | 136 | 150 |
| PCP0775 | 4507648 | NM_003289 | Cytoplasm | 4507648\|ref\|NM_003289.1 Homo sapiens tropomyosin 2 (beta) (TM2), mRNA | | | | | |
| 148, 149 | 4507648 | | | Tropomysin | Tropomosin | 521.5 | 6.10E− | 48 | 284 |
| | | | | | | | 168 | | |
| | 4507648 | | | bZIP | bZIP transcription factor | 10.3 | 0.011 | 33 | 66 |
| | 4507648 | | | OEP | Outer membrane efflux protein | −36.6 | 0.21 | 50 | 220 |
| | 4507648 | | | STAT prot | STAT protein, protein interaction dom | −49 | 0.49 | 102 | 206 |
| | 4507648 | | | Troponin | Troponin | −33 | 0.57 | 145 | 282 |
| | 4507648 | | | Hanta nucleocap | Hantavirus nucleocapsid protein | 0.1 | 0.77 | 90 | 119 |
| | 4507648 | | | M | M protein repeat | 11.9 | 0.79 | 223 | 243 |
| | 4507648 | | | Semialdhyde dh | Semialdehyde dehydrogenase, NAD bindi | 2 | 0.87 | 132 | 155 |
| PCP0791 | 7959918 | AF116710 | Cytoplasm | 7959918\|gb\|AF116710.1\|AF116710 Homo sapiens PRO2640 mRNA, complete cds. Also: | | | | | |
| | | | | ribosomal protein S14 (RPS14), mRNA | | | | | |
| 150 | 7959918 | | | Ribosomal S11 | Ribosomal protein S11 | 234.9 | 1.70E−73 | 29 | 147 |
| PCP0390 | 13529151 | NM_020169 | Cytoplasm | 13529151\|gb\|BC005346.1\|BC005346 Homo sapiens, latexin protein, clone MGC:12439, | | | | | |
| 151, 152 | 13529151 | | | mRNA, complete cds | | | | | |
| PCP0771 | 13938352 | NM_001007 | Cytoplasm | 13938352\|gb\|BC007308.1\|BC007308 Homo sapiens, Similar to RIKEN cDNA 1110033J19 | | | | | |
| 153, 154 | 13938352 | | | gene, clone MGC:15711, mRNA, complete cds. Also: risbosomal protein S4, linked (RPS4X), | | | | | |
| | | | | mRNA | | | | | |
| | 13938352 | | | Ribosomal S4e | Ribsosomal family S4e | 226.1 | 2.70E−65 | 29 | 134 |
| PCP0666 | 14015 | X55654 | Cyto/Nuc | 14015\|em\|X55654.1\|MTHSCOXII H.sapiens mitochondrial coxII mRNA for | | | | | |
| | | | | cytochrome C oxidase II | | | | | |
| 155 | 14015 | | | COX2 | Cytochrome C oxidase subunit II, peripl | 309.3 | 2.40E−90 | 54 | 185 |
| | 14015 | | | COX2 TM | Cytochrome C oxidase subunit II, transm | 63.1 | 1.60E−17 | 1 | 42 |
| PCP0415 | 517176 | NM_006106 | Cyto/Nuc | 517176\|emb\|X80507.1\|HSYAP65 H.sapiens YAP65.EST: 11945539\|gb\|BF671644.1\|BF671644 | | | | | |
| 156, 157 | 517176 | | | 6021152396F1 NIH_MGC_81 Homo sapiens cDNA clone IMAGE:42937 (plus/plus) | | | | | |
| | 517176 | | | WW | WW domain | 53.7 | 2.10E−13 | 173 | 202 |
| PCP0710 | 14042154 | NM_017647 | Nuclear | 14042154\|dbj\|AK027463.1\|AK027463 Homo sapiens cDNA FLJ14557 fis, clone NT2RM2001896, | | | | | |
| 158, 159 | 14042154 | | | weakly similar to CELL DIVISION PROTEIN FTSI. Also: | | | | | |
| | | | | mitochondrial coxII mRNA for cytochrome C oxidase II subunit | | | | | |
| | 14042154 | | | FtsJ | FtsJ-like methyltransfersae | 275.6 | 3.40E.80 | 24 | 202 |
| | 14042154 | | | COX2 TM | Cytochrome C oxidase subunit II, trans | 25.5 | 5.50E−07 | 458 | 493 |
| | 14042154 | | | Nol1 Nop2 Sun | NOL1/NOP2/sun family | 1.6 | 0.68 | 49 | 76 |
| | 14042154 | | | QRPTase | Quinolinate phosphoribosyl transferase | 2.1 | 0.84 | 326 | 335 |

TABLE 2

| | | | | | | | | |
|---|---|---|---|---|---|---|---|---|
| GI# | NM | classification | domain | description | Score | E | seq-f | seq-t |

| PCP0620 | 4929680 | NM_013237 | Cytoplasm | 492980\|gb\|AF151864.1\|AF151864 Homo sapiens CGI-106 protein mRNA, complete cds and gb\| | | | | | |
|---|---|---|---|---|---|---|---|---|---|
| 160, 161 | 4929680 | | | AF112203.1\|AF112203 Homo sapiens PX19 protein mRNA, complete | | | | | |
| PCP0714 | 11640567 | NM_000969 | Cytoplasm | 11640567\|gb\|AF113210.1\|AF112310 Homo sapiens MSTP030 mRNA, complete cds. Also: | | | | | |
| | | | | ribosomal protein L5 (RPL5), mRNA | | | | | |
| 162, 163 | 11640567 | | | Ribosomal L18p | Ribosomal L18p/L5e family | 261.9 | 4.50E−76 | 26 | 173 |
| PCP05619 | 14249863 | NM_013237 | Cytoplasm | 14249863\|bg\|BC008307.1\|BC008307 Homo sapiens, px19-like protein, clone MGC:15214, | | | | | |
| 164, 165 | 14249863 | | | mRNA, complete cds | | | | | |
| PCP0513 | 14249920 | AY029066 | Unknown | 14249920\|gb\|BC008341.1\|BC008341 Homo sapiens, clone MGC:15852, mRNA, compete cds | | | | | |
| 166, 167 | 14249920 | | | EST: 13285972\|gb\|BG392524.1\|BC392524 6024107729F1 NIH_MGC_92 | | | | | |
| | | | | Homo sapiens cDNA clone IMAGE:4539727 5′ (plus/plus) | | | | | |
| | 14249920 | | | | | | | | |
| PCP0708 | 12053382 | NM_014585 | Membrane | 12053382\|emb\|AL136944.1\|HSM801908 Homo sapiens mRNA; cDNA DKFZp58610624 | | | | | |
| | | | | (from clone DKFZp58610624); complete cds. Also: SLC11A3 iron transporter | | | | | |
| 168, 169 | 12053382 | | | xan ur permease | Permease family | −189.7 | 0.47 | 92 | 426 |
| | 12053382 | | | DUF33 | Domain of unknown function DUF33 | 1.4 | 0.97 | 54 | 77 |
| PCP0551 | 13650139 | NM_007043 | Nuclear | 13650139\|ref\|XM_006702.3\|Homo sapiens HIV-1 rev binding protein 2 (HRB2), | | | | | |
| 170, 171 | 13650139 | | | mRNAEST: 14000799\|gb\|BG721612.1\|BG721612 602695736F1 NIH_MGC_97 | | | | | |
| | | | | Homo sapiens cDNA clone IMAGE:4827704 5′ (plus/plus) | | | | | |
| PCP0803 | 14249297 | M28016 | Extracellular | 1424929\|ref\|NM_032702.1 Homo sapiens hypothetical protein MGC2724 (MGC2724), mRNA. | | | | | |
| 172 | 14249297 | | | A2M | Alpha 2-macroglobulin family | 59.4 | 7.10E−19 | 1 | 86 |
| PCP0774 | 182734 | K00650 | Nuclear | 182734 K00650.1\|HUMFOS Human fos proto-oncogene (c-fos), complete cds | | | | | |
| 173, 174 | 182734 | | | bZIP | bZIP transcription factor | 35.3 | 9.40E−10 | 135 | 199 |
| | 182734 | | | bZIP Maf | bZIP Maf transcription factor | −69.5 | 0.083 | 180 | 215 |
| | 182734 | | | DegT DnrJ EryC1 | DegT/DnrJ/EryC1/StrS family | −244.5 | 0.9 | 20 | 260 |
| PCP0615 | 291872 | NM_005180 | Nuclear | 291872\|gb\|L13689.1\|HUMBMI1X Human prot-oncogene (BMI-1) mRNA, complete cds | | | | | |
| 175, 176 | 291872 | | | zf-C3HC4 | Zinc finger, C3HC4 type (RING finger) | 50.5 | 1.60E−15 | 18 | 56 |
| | 291872 | | | PHD | PHD-finger | −20.5 | 0.73 | 17 | 59 |
| PCP0825 | 414929 | NM_003467 | Membrane | 414929 L06797.1\|HUMGPCR Human clone (L5) orphan G protein-coupled recpeter mRNA, | | | | | |
| 177, 178 | 414929 | | | complete cds | | | | | |
| PCP0585 | 476104 | NM_004882 | Nuclear | 476104\|gb\|U03644.1\|HSU03644 Human recepin mRNA, complete cds and ref\|XM_002455.2\| | | | | | |
| 179, 180 | 476104 | | | Homo sapiens CBF1 interacting corepressor (CIR), mRNA | | | | | |
| PCP0857 | 3860076 | NM_006418 | Extracellular | 3860076 AF097021 Homo sapiens GW112 protein (GW112) mRNA | | | | | |
| 181, 182 | 3860076 | | | | | | | | |
| PCP0711 | 4503492 | NM_001964 | Nuclear | 4503492\|ref\|NM_001964.1 Homo sapiens early growth response 1 (EGR1), mRNA. | | | | | |
| | | | | Also: zinc finger protein and transcription factor ETR103 | | | | | |
| 183, 184 | 4503492 | | | zf-C2H2 | Zinc finger, C2H2 type | 91.3 | 1.00E−24 | 396 | 418 |
| | 4503492 | | | hormone | Somatotropin hormone family | 2 | 0.11 | 150 | 173 |
| | 4503492 | | | zf-BED | BED zinc finger | −4.2 | 0.22 | 357 | 391 |
| | 4503492 | | | thiored | Thioredoxin | 3.1 | 0.7 | 94 | 118 |
| PCP0841 | 4583651 | NM_005977 | Nuclear | 4583651\|emb\|AJ010346.1\|HSA010346 Homo sapiens mRNA for RING-H2 protein FNF6, | | | | | |
| | | | | alternative exon 1a | | | | | |
| 185, 186 | 4583651 | | | zf-C3HC4 | Zinc finger, C3HC4 type (RING finger) | 27.5 | 1.80E−08 | 632 | 672 |
| | 4583651 | | | PA28 beta | Proteasome activator pa28 beta subuni | −84.6 | 0.37 | 368 | 517 |
| | 4583651 | | | PHD | PHD-finger | −19.9 | 0.63 | 631 | 675 |
| | 4583651 | | | RNA pal omega | RNA polymerase omega subunit | −16.9 | 0.93 | 373 | 455 |
| PCP0849 | 11433150 | NM_001870 | Extracellular | 11433150 XM_0030081\|Homo sapiens carboxypeptidase A3 (mast cell) (CPA3), mRNA | | | | | |
| 187, 188 | 11433150 | | | Zn carbOpept | Zinc carboxypeptidase | 568.8 | 1.80E− | 119 | 400 |
| | | | | | | | 168 | | |
| | 11433150 | | | Propep M14 | Carboxypeptidase activation peptide | 161.4 | 7.70E−46 | 25 | 104 |
| PCP0814 | 12053078 | NM_030817 | Membrane | 12053078\|emb\|AL136783.1\|HSM801751 Homo sapiens mRNA; cDNA DKFZp434F0318 | | | | | |
| | | | | (from clone DKFZp434F0318); complete cds | | | | | |
| 189, 190 | 12053078 | | | MotA ExbB | MotA/TolQ/ExbB proton channel family | −52.8 | 0.11 | 3 | 125 |
| | 12053078 | | | CMD | Carboxymuconolactone decarboxylase | −28.6 | 0.7 | 17 | 106 |
| | 12053078 | | | Tropomyosin | Tropomyosin | 1.3 | 0.71 | 198 | 229 |
| PCP0530 | 12654422 | NM_000996 | Cytoplasm | 12654422\|gb\|BC001037.1\|BC001037 Homo sapiens, robosomal protein L35a, clone MGC:1639, | | | | | |
| | | | | mRNA | | | | | |
| 191, 192 | 12654422 | | | Ribosomal L35Ae | Ribosomal protein L35Ae | 269 | 3.10E−78 | 5 | 104 |
| PCP0798 | 12732572 | XM_165833 | Extracellular | 12732572\|ref\|XM_004467.2 Homo sapiens coagulation factor XIII, A1 polypeptide (F13A1), | | | | | |
| | | | | mRNA | | | | | |
| 193, 194 | 12732572 | | | Transglutamin C | Transglutaminase family | 359.3 | 2.10E− | 503 | 728 |
| | | | | | | | 105 | | |
| | 12732572 | | | Tansglutamin N | Transglutaminase family | 225 | 5.50E−65 | 46 | 167 |
| | 12732572 | | | Tansglut core | Transglutaminase-like superfamily | 187.4 | 1.20E−53 | 310 | 399 |
| | 12732572 | | | Terpene synth | Terpene synthase family | 1.6 | 0.4 | 56 | 67 |
| PCP0581 | 13647040 | NM_015865 | membrane | 13647040\|ref\|XM_012742.2\|Homo sapiens solute carrier family 14 (urea transporter), | | | | | |
| 195, 196 | 13647040 | | | member 1 (Kidd blood group) (SLC14A1), mRNAEST: 13040853\|gb\| | | | | | |
| | | | | BG287225.1\|BG287225 602381952F1 NIH_MGC_92 Homo sapiens cDNA clone | | | | | |
| | | | | IMAGE:4499388 5′ (plus/plus) | | | | | |
| | 13647040 | | | DUF6 | Integral membrane protein DUF6 | −28.1 | 0.8 | 160 | 276 |
| PCP0750 | 13647324 | NM_001964 | Nuclear | 13647324\|ref\|XM_0064063.2 Homo sapiens early growth response 1 (EGR1), mRNA | | | | | |
| 197, 198 | 13647324 | | | zf-C2H2 | Zinc finger, C2H2 type | 91.3 | 1.00E−24 | 396 | 418 |
| | 13647324 | | | hormone | Somatotropin hormone family | 2 | 0.11 | 150 | 173 |
| | 13647324 | | | zf-BED | BED zinc finger | −4.2 | 0.22 | 357 | 391 |
| | 13647324 | | | thiored | Thioredoxin | 3.1 | 0.7 | 94 | 118 |
| PCP0810 | 14017398 | AY029066 | Extracellular | 14017398\|gb\|AY029066.1\|Homo sapiens Humanin (HN1) mRNA, complete cds. Also: 7020373\| | | | | | |
| 199, 200 | 14017398 | | | dbj\|AK000348.1\|AK000348 Homo sapiens cDNA FLJ20341 fis, clone HEP13116, | | | | | |
| | | | | highly similar to D43949 Human mRNA for KIAA0082 gene | | | | | |
| | 14017398 | | | | | | | | |
| PCP0864 | 14736688 | NM_002343 | Extracellular | 14736688\|ref\|XM_030418.1\|Homo sapiens lactotransferrin (LTF), mRNA | | | | | |
| 201, 202 | 14736688 | | | transferrin | Transferrin | 1219.8 | 0 | 365 | 696 |
| | 14736688 | | | Mago nashi | Mago Mashi protein | 1.6 | 0.66 | 584 | 603 |
| PCP0792 | 6808606 | NM_013281 | Membrane | 680606\|gb\|AF169677.1\|AF169677 Homo sapiens lecuine-rich repeat transmembrane | | | | | |
| | | | | protein FLRT3 (FLRT3) mRNA, complete cds | | | | | |
| 203, 204 | 6808606 | | | LRR | Leucine Rich Repeat | 134.4 | 1.10E−37 | 272 | 295 |
| | 6808606 | | | LRR | Leucine rich repeat C-terminal domain | 55.8 | 4.90E−14 | 305 | 356 |
| | 6808606 | | | LRRNT | Leucine rich repeat N-terminal domain | 27.4 | 1.80E−05 | 30 | 57 |
| | 6808606 | | | fn3 | Fibronectin type III domain | 11.1 | 0.011 | 405 | 485 |
| PCP0722 | 7019382 | NM_013281 | Membrane | 7019382\|ref\|NM_013281.1\|Homo sapiens fibronectin leucine rich transmembrane protein | | | | | |
| | | | | 3 (FLRT3), mRNA | | | | | |
| 205, 206 | 7019382 | | | LRR | Leucine Rich Repeat | 134.4 | 1.10E−37 | 272 | 295 |
| | 7019382 | | | LRRCT | Leucine rich repeat C-terminal domain | 55.8 | 4.90E−14 | 305 | 356 |
| | 7019382 | | | LRRNT | Leucine rich repeat N-terminal domain | 27.4 | 1.80E−05 | 30 | 57 |
| | 7019382 | | | fn3 | Fibronectin type III domain | 11.1 | 0.011 | 405 | 485 |
| PCP0642 | 10190747 | NM_020974 | Extracellular | 10190747\|ref\|NM_020974.1\|Homo sapiens CEGP1 protein (CEPG1), mRNA | | | | | |
| 207, 208 | 10190747 | | | EGF | EGF-like domain | 228.3 | 5.70E−66 | 407 | 442 |
| | 10190747 | | | CUB | CUB domain | 70.3 | 2.00E−18 | 809 | 918 |
| | 10190747 | | | granulin | Granulin | 7.5 | 0.027 | 303 | 323 |
| | 10190747 | | | TIL | Trypsin Inhibitor like cysteine rich | −2 | 0.038 | 84 | 132 |
| | 10190747 | | | TNFR c6 | TNER/NGFR cysteine-rich region | 9 | 0.046 | 698 | 728 |
| | 10190747 | | | Keratin B2 | Keratin, high sulfur B2 protein | −67.5 | 0.047 | 111 | 242 |
| | 10190747 | | | S mold repeat | Dictyostelium (slime mold) repeat | 8.2 | 0.67 | 125 | 149 |
| PCP0740 | 13097704 | XM_028322 | Extracellular | 13097704\|gb\|BC003559.1\|BC003559 Homo sapiens, serine (or cysteine) proteinase inhibitor, | | | | | |
| 209, 210 | 13097704 | | | clade A (alpha-1 antiproteinase, antitrypsin), member 3, clone MGC:1813, complete cds | | | | | |
| | 13097704 | | | sepin | Serpin (serine protease inhibitor) | 735.2 | 1.50E− | 146 | 420 |
| | | | | | | | 218 | | |
| PCP0819 | 13173403 | AF339085 | Membrane | 13173403 AF339085 Homo sapiens NADH dehydrogenase subunit 5 (MTND5) mRNA, RNA 5 | | | | | |
| 211, 212 | 13173403 | | | oxidored ql | NADH-Ubiquinone/plastoquinone | 381.5 | 4.50E− | 134 | 420 |
| | | | | | (comp1) | | 112 | | |
| | 13173403 | | | oxidored ql N | NADH-Ubiquinone oxidoreductase | 110.2 | 2.10E−30 | 62 | 123 |
| | | | | | (comp | | | | |
| | 13173403 | | | Na Pi cotrans | Na⁺/Pi-cotransporter | −160.7 | 0.41 | 217 | 585 |
| | 13173403 | | | UPF0032 | MttB family UPF0032 | −95.9 | 0.44 | 273 | 483 |
| | 13173403 | | | ERG4 ERG24 | Ergosteral biosynthesis ERG4/ERG24 fa | −316.1 | 0.61 | 249 | 597 |
| | 13173403 | | | xan ur permease | Permease family | −195.1 | 0.72 | 209 | 536 |
| | 13173403 | | | ABC2 membrane | ABC-2 type transporter | −137.3 | 0.72 | 173 | 427 |
| | 13173403 | | | MVIN | Virulence factor MVIN | −260.3 | 0.8 | 63 | 529 |
| | 13173403 | | | sugar tr | Sugas (and other) transporter | −205.6 | 0.89 | 166 | 557 |
| | 13173403 | | | 7tm 3 | 7 transmembrane receptor (metabotropi | −165.1 | 0.93 | 239 | 436 |
| PCP0385 | 13632411 | NM_001099 | Membrane | 13632411\|ref\|XM_002837.3\|Homo sapiens acid phosphatase, prostate (ACPP), mRNA | | | | | |
| 213, 214 | 13632411 | | | acid phosphat | Histidine acid phosphatase | 539.3 | 1.40E− | 33 | 373 |
| | | | | | | | 159 | | |
| PCP0515 | 13642675 | NM_004342 | Cytoplasm | 13642675\|ref\|XM _01557.2\|Homo sapiens region containing NAG22 protein; caldesmon 1 | | | | | |
| 215, 216 | 13642675 | | | (LOC83043), mRNAEST: 10997856\|dbj\|AU137317.1\|AU137317 AU137317 PLACE1 | | | | | |
| | | | | Homo sapiens cDNA clone PLACE1006233 5′ (plus/plus) | | | | | |
| | 13642675 | | | Caldesmon | Caldesmon | 1059.1 | 0 | 1 | 538 |
| | 13642675 | | | G-gamma | GGL domain | 4.5 | 0.24 | 346 | 367 |
| | 13642675 | | | KIX | KIX domain | −19.2 | 0.75 | 255 | 319 |
| PCP0406 | 13643509 | NM_002343 | Extracellular | 13643509\|ref\|XM_010963.2\| Homo sapiens lactotransferrin (LTF), mRNA | | | | | |
| 217, 218 | 13643509 | | | transferrin | Transferrin | 269.2 | 1.00E−78 | 1 | 285 |
| | 13643509 | | | Mago nashi | Mago nashi protein | 1.6 | 0.66 | 173 | 192 |
| PCP0518 | 13644505 | NM_001723 | Nuclear | 13644505\|ref\|XM_004233.3\| Homo sapiens bullous pemphigoid antigen 1 (230/240kd) | | | | | |
| 219, 220 | 13644505 | | | (BPAG1), mRNAEST: 11592016\|gb\|BF508718.1\|BF508718 UI-H-B14-aoq-a-04 0-ULs1 NCI_ | | | | | |
| | | | | CGAP_Sub8 Homo sapiens cNDA clone (plus/minus) | | | | | |
| | 13644505 | | | Plectin repeat | Plectin repeat | 310.7 | 9.30E−91 | 2553 | 2597 |
| | 13644505 | | | spectrin | Spectrin repeat | 42.4 | 1.40E−11 | 935 | 961 |
| | 13644505 | | | bZIP | bZIP transcription factor | 14.9 | 0.00058 | 1608 | 1638 |
| | 13644505 | | | Tropomyosin | Tropomyosin | 7.4 | 0.0081 | 1831 | 1859 |
| | 13644505 | | | M | M protein repeat | 18.5 | 0.0082 | 1826 | 1846 |
| | 13644505 | | | ldh C | lactate/malate dehydrogenase, alpha/b | 8.7 | 0.02 | 1527 | 1552 |
| | 13644505 | | | Myc-LZ | Myc leucine zipper domain | 17.1 | 0.021 | 1595 | 1630 |
| | 13644505 | | | SH3 | SH3 domain | −6.9 | 0.03 | 564 | 616 |
| | 13644505 | | | PCNA | Proliferating cell nuclear antigen, N | −62.2 | 0.16 | 779 | 874 |
| | 13644505 | | | HlyC | RTX toxin acyltransferase family | 3.4 | 0.27 | 414 | 423 |
| | 13644505 | | | B | B domain | −4.8 | 0.37 | 1616 | 1669 |
| | 13644505 | | | RPEL | RPEL repeat | 5.1 | 0.49 | 1502 | 1527 |
| | 13644505 | | | Ribosomal S3 N | Ribosomal protein S3 N-terminal doma | 3 | 0.5 | 2123 | 2149 |
| | 13644505 | | | ERM | Ezrin/radixin/moesin family | −226.2 | 0.58 | 1170 | 1424 |
| | 13644505 | | | Lipoprotein 7 | Adhesin lipoprotein | −131 | 0.59 | 1376 | 1820 |
| | 13644505 | | | FCH | Fes/CIP4 homology domain | −21.2 | 0.73 | 1773 | 1863 |
| | 13644505 | | | Paramyxo P | Paramyxovirus P phosphoprotein | −335 | 0.75 | 407 | 872 |
| | 13644505 | | | SNAP-25 | SNAP-25 family | −57.5 | 0.79 | 246 | 442 |
| | 13644505 | | | enolase | Enol-ase | 0.7 | 0.8 | 1156 | 1176 |
| | 13644505 | | | PGAM | Phosphoglycerate mutase family | −130.9 | 0.86 | 169 | 357 |
| | 13644505 | | | RecX | RecX family | −38.3 | 0.9 | 272 | 393 |
| | 13644505 | | | HDV ag | Hepatitis delta virus delta antigen | −53.7 | 0.96 | 1167 | 1356 |
| PCP0623 | 13644568 | NM_015384 | Nuclear | 13644568\|ref\|XM_003877.3\| Homo sapiens IDN3 protein (IDN3), mRNAEAST: | | | | | |
| 221, 222 | 13644568 | | | 14213250\|gb\|BG862712.1 BM862712 602796442F1 NIH_CGAP_Mam4 | | | | | |
| | | | | Mus musculus cDNA clone IMAGE:4917443 5′ (plus/plus) | | | | | |
| | 13644568 | | | MIF4G | MIF4G domain | −20.5 | 0.12 | 481 | 743 |
| | 13644568 | | | Rep | Replication protein | 4.7 | 0.15 | 977 | 993 |
| | 13644568 | | | thymidylat syn | Thymidylate synthase | 2.8 | 0.53 | 461 | 479 |
| | 13644568 | | | LMWPc | Low molecular weight phosphotyrosine | −53.2 | 0.61 | 450 | 574 |
| | 13644568 | | | FliG-C | FliG C-terminal domain | −50.6 | 0.68 | 223 | 335 |
| | 13644568 | | | ATP-synt DE | ATP synthase, Delta/Epsilon chain, lo | 3.3 | 0.76 | 300 | 347 |
| | 13644568 | | | interferon | Interferon alpha/beta domain | 2 | 1 | 850 | 861 |
| PCP0641 | 14740173 | NM_025226 | Membrane | 14740173\|re\|XM_017806.2\| Homo sapiens MSTP032 protein (MSTP032), mRNA | | | | | |
| 223, 224 | 14740173 | | | | | | | | |
| PCP0577 | 14765929 | NM_015865 | Membrane | 14765929\|ref\|XM_012742.3 Homo sapiens solute carrier family 14 (urea trasnporter), | | | | | |
| | | | | member 1 (Kidd blood group) (SLC14A1), mRNA | | | | | |
| 225, 226 | 14765929 | | | DUF6 | Integral membrane protein DUF6 | −28.1 | 0.8 | 160 | 276 |
| PCP0709 | 179295 | L08441 | Cytoplasm | 179295\|gb\|L08441.1\|HUMAUTONH Human autonomously replicating sequence (ARS) mRNA | | | | | |
| 227, 228 | 179295 | | | COX3 | Cytochrome c oxidase subunit III | 649.5 | 9.00E− | 6 | 261 |
| | | | | | | | 193 | | |
| PCP0474 | 179518 | NM_001723 | Nuclear | 179518\|gb\|M63618.1\|HUMBPA Human bullous pemphigoid antigen mRNA, complete cds | | | | | |
| 229, 230 | 179518 | | | Plectin repeat | Plectin repeat | 310.7 | 9.30E−91 | 1999 | 2043 |
| | 179518 | | | bZIP | bZIP transcription factor | 11.9 | 0.004 | 1054 | 1084 |
| | 179518 | | | Tropomyosin | Tropomyosin | 7.4 | 0.0081 | 1277 | 1305 |
| | 179518 | | | M | M protein repeat | 18.5 | 0.0082 | 1272 | 1292 |
| | 179518 | | | ldh C | lactate/malate dehydrogenase, alpha/b | 8.7 | 0.02 | 973 | 998 |
| | 179518 | | | SH3 | SH3 domain | −6.9 | 0.03 | 10 | 62 |
| | 179518 | | | Myc-LZ | Myc leucine zipper domain | 14.7 | 0.06 | 1041 | 1076 |
| | 179518 | | | PCNA | Proliferating cell nuclear antingen, N | −62.2 | 0.16 | 225 | 320 |
| | 179518 | | | spectrin | Spectrin repeat | 5.5 | 0.24 | 381 | 407 |
| | 179518 | | | B | B domain | −4.8 | 0.37 | 1062 | 1115 |
| | 179518 | | | RPEL | RPEL repeat | 5.1 | 0.49 | 948 | 973 |
| | 179518 | | | Ribosomal S3 N | Ribosomal protein S3, N-terminal doma | 3 | 0.5 | 1569 | 1595 |
| | 179518 | | | ERM | Ezrin/radixin/moesin family | −226.2 | 0.58 | 616 | 870 |
| | 179518 | | | Lipoprotein 7 | Adhesion lipoprotein | −131 | 0.59 | 822 | 1266 |
| | 179518 | | | FCH | Fes/CIP4 homology domain | −21.2 | 0.73 | 1219 | 1309 |
| | 179518 | | | enolase | Enol-ase | 0.7 | 0.8 | 602 | 622 |
| | 179518 | | | HDV ag | Hepatitis delta virus delta antigen | −53.7 | 0.96 | 613 | 802 |
| PCP0427 | 311380 | NM_030752 | Cytoplasm | 311380\|emb\|X52882.1\|HSTCP1 Human t-complex polypetide 1 gene | | | | | |
| 231, 232 | 311380 | | | cpn60 TCP1 | TCP-1/cpn60 chaperonin family | 808 | 1.80E− | 28 | 535 |
| | | | | | | | 240 | | |
| | 311380 | | | ATP-synt B | ATP synthase B/B′ CF(0) | 4 | 0.36 | 302 | 337 |
| PCP0425 | 3170185 | NM_005869 | Nuclear | 3170185\|gb\|AF039693.1\|AF039693 Homo sapiens unknown protein mRNA, complete cdsEST: | | | | | |
| 233, 234 | 3170185 | | | 5234657\|bg\|AI768148.1\|AI768148 wg81f10.x1 Soares NSF F8 9W OT PA P S1 Homo sapiens | | | | | |
| | | | | cDNA cloneIMAGE:2371531 3′ similar to TR:O60530 O60530 HYPOTHETICAL | | | | | |
| | | | | 38.4 KD PROTEIN (plus/minus) | | | | | |
| | 3170185 | | | Flu PB1 | Influenze RNA-dependent RNA polymera | −0.1 | 0.76 | 35 | 49 |
| PCP0495 | 5454137 | NM_006311 | Nuclear | 5454137\|ref\|NM_006311.1 Homo sapiens nuclear receptor co-repressor 1 (NCOR1), mRNA | | | | | |
| 235, 236 | 5454137 | | | myb DNA- | Myb-like DNA-binding domain | 98.9 | 4.20E−28 | 625 | 670 |
| | | | | binding | | | | | |
| | 5454137 | | | RNA pol A | RNA polymerase alpha subunit | 2 | 0.071 | 307 | 341 |
| | 5454137 | | | spectrin | Spectrin repeat | 5.2 | 0.29 | 301 | 327 |
| PCP0384 | 13173405 | AF339086 | Cytoplasm | 13173405\|gb\|AF339086.1\|AF339086 Homo sapiens NADH dehydrogenase subunit 5 | | | | | |
| | | | | (MTND5) mRNA, RNA 4, complete cds; mitochondrial gene for mitochondrial product | | | | | |
| 237, 238 | 13173405 | | | oxidored q1 | NADH-Ubiquinone/plastoquinone | 381.5 | 4.50E− | 134 | 420 |
| | | | | | (comp1) | | 112 | | |
| | 13173405 | | | oxidored q1 N | NADH-Ubiquinone oxidoreductase (comp | 110.2 | 2.10E−30 | 62 | 123 |
| | 13173405 | | | Na Pi contrans | Na⁺/Pi-cotransporter | −160.7 | 0.41 | 217 | 585 |
| | 13173405 | | | UPF0032 | MttB family UPF0032 | −95.9 | 0.44 | 273 | 483 |
| | 13173405 | | | ERG4 ERG24 | Ergosterol biosynthesis ERG4/ERG24 fa | −316.1 | 0.61 | 249 | 597 |
| | 13173405 | | | xan ur permease | Permease family | −195.1 | 0.72 | 209 | 536 |
| | 13173405 | | | ABC2 membrane | ABC-2 type transporter | −137.3 | 0.72 | 173 | 427 |
| | 13173405 | | | MVIN | Virulence factor MVIN | −260.3 | 0.8 | 63 | 529 |
| | 13173405 | | | sugar tr | Sugar (and other) transporter | −205.6 | 0.89 | 166 | 557 |
| | 13173405 | | | 7tm 3 | 7 Transmembrane receptor (metabotropi | −165.1 | 0.93 | 239 | 436 |

1. A method of determining the presence of prostate cancer cells in a sample comprising nucleic acid, comprising:
contacting said sample with a polynucleotide probe under conditions effective for said probe to hybridize specifically to a target nucleic acid in said sample,
detecting the amount of hybridization between said probe and target nucleic acid, and
determining by said hybridization whether said target nucleic acid is differentially-regulated in said sample, whereby the presence of a differentially-regulated target nucleic acid indicates that said sample comprises cancer cells,
wherein said probe comprises a polynucleotide sequence which is selected from Table 1 or 2, a polynucleotide having 95% sequence identity or more to a polynucleotide sequence selected from Table 1 or 2, effective specific fragments thereof, or complements thereto.

2. A method of claim 1, wherein said determining comprises:
comparing the amount of hybridization in said sample with the amount of hybridization of said probe in a second sample comprising normal prostate.

3. A method of claim 1, wherein said probe is a contiguous sequence of at least 16 nucleotides selected from a polynucleotide of Table 1 or 2, or a complement thereto.

4. A method of claim 1, wherein said detecting is performed by Northern blot analysis, polymerase chain reaction (PCR), reverse transcriptase PCR, RACE PCR, or in situ hybridization.

5. A method for diagnosing a prostate cancer in a sample comprising prostate tissue, comprising:
determining the number of target genes which are differentially-regulated in said sample, wherein said target genes are selected from Table 1 or 2, or, a gene represented by a sequence having 95% sequence identity or more to a sequence selected from Table 1 or 2,
wherein said genes are differentially-regulated in prostate cancer, and
whereby said number is indicative of the probability that said sample comprises prostate cancer.

6. A method of claim 5, wherein said determining is performed by Northern blot analysis, polymerase chain reaction (PCR), reverse transcriptase PCR, RACE PCR, or in situ hybridization using a polynucleotide probe which is selected from Table 1 or 2, a polynucleotide having 95% sequence identity or more to a sequence set forth in Table 1 or 2, effective specific fragments thereof, or complements thereto.

7. A method of claim 5, wherein said determining is performed by:
contacting said sample with a polynucleotide probe under conditions effective for said probe to hybridize specifically to a target nucleic acid in said sample, and
detecting the amount of hybridization between said probe and target nucleic acid, and comparing the amount of hybridization in said sample with the amount of hybridization of said probe in a second sample comprising normal prostate tissue.

8. A method of clam 5, wherein said probe is a contiguous sequence of at least 16 nucleotides selected from a polynucleotide listed in Table 1 or 2, or a complement thereto.

9. A method of assessing a therapeutic or preventative intervention in a subject having a prostate cancer, comprising,
detecting the expression levels of differentially-regulated genes, wherein the target genes comprise a gene which is represented by a polynucleotide listed in Table 1 or 2 of claim 21, or, a gene represented by a sequence having 95% sequence identity thereto.

10. A method of claim 9, wherein said detecting is performed by Northern blot analysis, polymerase chain reaction (PCR), reverse transcriptase PCR, RACE PCR, or in situ hybridization using a polynucleotide probe which is selected from Table 1 or 2, a polynucleotide having 95% sequence identity or more to a polynucleotide selected from Table 1 or 2, effective specific fragments thereof, or complements thereto.

11. A method for identifying agents that modulate the expression of target polynucleotides differentially-regulated in prostate cancer cells, comprising,
contacting a prostate cell population with a test agent under conditions effective for said test agent to modulate the expression of a target polynucleotide in said cell population, and
determining whether said test agent modulates said target polynucleotide expression, wherein said target polynucleotide is selected from Table 1 or 2 of claim 21, a polynucleotide having 95% sequence identity thereto, effective specific fragments thereof, or complements thereto, and said polynucleotide is differentially-regulated in a prostate cancer.

12. A method of claim 1 1, wherein said agent is an antisense polynucleotide to a target polynucleotide sequence selected from Table 1 or 2 and which is effective to inhibit translation of said target polynucleotide.

13. A method for identifying agents that modulate a biological activity of a polypeptide differentially-regulated in prostate cancer cells, comprising,
contacting a polypeptide differentially-regulated in prostate cancer cells with a test agent under conditions effective for said test agent to modulate a biological activity of said polypeptide, and
determining whether said test agent modulates said biological activity, wherein said polypeptide is coded for by a polynucleotide listed in Table 1 or 2, of claim 21, a polynucleotide having 95% sequence identity thereto, effective specific fragments thereof, or complements thereto, and said polynucleotide is differentially-regulated in a prostate cancer.

14. A method of treating prostate cancer, comprising,
administering to a subject in need thereof a therapeutic agent which is effective for regulating expression of at least one gene selected from Table 1 or 2 of claim 21, wherein said gene is differentially-regulated in said cancer.

15. A method of claim 14, wherein said agent is an antibody or an antisense which is effective to inhibit translation of said gene.

16. A method of diagnosing a prostate cancer comprising:
assessing the expression of at least one gene selected from Table 1 or 2 of claim 21, wherein said gene is differentially-regulated in said cancer.

17. A method of claim 16, wherein assessing is:
measuring mRNA expression levels of said or measuring the expression levels of polypeptide coded for by said gene.

18. A method of claim 16, wherein said assessing detecting is performed by: Northern blot analysis, polymerase chain reaction (PCR), reverse transcriptase PCR, RACE PCR, or in situ hybridization, and
using a polynucleotide probe having a nucleotide sequence selected from a polynucleotide listed in Table 1 or 2, a polynucleotide having 95% sequence identity thereto, effective specific fragments thereof, or complements thereto.

19. A method of retrieving prostate cancer differentially-regulated gene sequences from a computer-readable medium, comprising:
selecting a gene expression profile that specifies that said gene is differentially-regulated in a prostate cancer, and retrieving prostate cancer differentially-regulated gene sequences,
where the gene sequences consist of the sequences of polynucleotides listed in Tables 1 or 2 of claim 21, a polynucleotide having 95% sequence identity thereto, effective specific fragments thereof, or complements thereto.

20. An ordered array of polynucleotide probes for detecting the expression of differentially-regulated prostate cancer genes in a sample, comprising:
polynucleotide probes associated with a solid support, wherein each probe is specific for a different differentially-regulated prostate cancer gene, and the probes comprise a nucleotide sequence selected the polynucleotides listed in from Table 1 or 2 of claim 21, or a complement thereto.

21. A computer-readable storage medium, consisting essentially of, one or more differentially-regulated cancer prostate genes which are selected from Table 1 or 2, a polynucleotide having 95% sequence identity thereto, effective specific fragments thereof, or complements thereto, and said polynucleotide is differentially-regulated in said prostate cancer.
